SEQUENCE LISTING (8 sequences)

SEQ ID NO: 1 - protein, 11 aa, Artificial
  GnRH-monomer
  MISC_FEATURE (1)..(1): “E” on pos. 1 is pyroGlutamine
EHWSYGLRPGC

SEQ ID NO: 2 - protein, 21 aa, Artificial
  GnRH-tandem
  MISC_FEATURE (1)..(1): “E” on pos. 1 is pyroGlutamine
EHWSYGLRPGQHWSYGLRPGC

SEQ ID NO: 3 - protein, 17 aa, Artificial
  Gastrin-like peptide
  MISC_FEATURE (1)..(1): “E” on pos. 1 is pyroGlutamine
EGPWLEEEEEAYGWMDF

SEQ ID NO: 4 - protein, 19 aa, Artificial
  Peptide derived from gastrin
  MISC_FEATURE (1)..(1): “E” on pos. 1 is pyroGlutamine
EGPWLEEEECQGPWLEEEE

SEQ ID NO: 5 - protein, 20 aa, Artificial
  Gastrin derived peptide
  MISC_FEATURE (1)..(1): “E” on pos. 1 is pyroGlutamine
  MISC_FEATURE (19)..(20): “C” on pos. 19 is bound to peptide with sequence EGPWLEEEEQGPWLEEEECK;  “K” on pos. 20 is bound to an amide
EGPWLEEEEQGPWLEEEECK

SEQ ID NO: 6 - protein, 18 aa, Artificial
  Gastrin derived peptide
  MISC_FEATURE (1)..(1): “E” on pos. 1 is pyroGlutamine
  MISC_FEATURE (18)..(18): “K” on pos. 18 is biotinylated Lysin and bound to an amide
EGPWLEEEEEAYGWMDFK

SEQ ID NO: 7 - protein, 10 aa, Artificial
  Mono gastrin
  MISC_FEATURE (1)..(10): “E” on pos. 1 is pyroGlutamine; “C” on pos. 10 is amidated
EGPWLEEEEC

SEQ ID NO: 8 - protein, 19 aa, Artificial
  Tandem gastrin
  MISC_FEATURE (1)..(19): “E” on pos. 1 is pyroGlutamine; “C” on pos. 19 is amidated
EGPWLEEEEQGPWLEEEEC

(19) United States
(12) Patent Application Publication
(10) Pub. No.: US 2007/0065428 A1
(43) Pub. Date: Mar. 22, 2007

(54) Early B-cell detection for selecting vaccines

(75) Inventors: Gerardus Josephus Adrianus Arkesteijn, Utrecht (NL); Everard Johannes Hensen, Utrecht (NL); Antonio Scibelli, Napoli (IT); Robbert Gerrit Van Der Most, Odijk (NL); Robbert Hans Meloen, Lelystad (NL); Jouwert Anne Turkstra, Dronten (NL)

Correspondence Address: TRASK BRITT, P.O. BOX 2550, SALT LAKE CITY, UT, 84110, US

(73) Assignee: Pepscan Systems B.V., Utrecht (NL)

(21) Appl. No.: 11/502,744

(22) Filed: Aug. 11, 2006

Related U.S. Application Data
(63) Continuation of application No. PCT/NL05/00101, filed on Feb. 11, 2005.

(30) Foreign Application Priority Data
Feb. 12, 2004 (EP) 04075439.2

(51) Int. Cl. A61K 39/395; G01N 33/567

(52) U.S. Cl. 424/133.1; 435/7.2

(57) ABSTRACT

The present invention discloses an affinity-binding assay comprising a particle having at least four copies of a target molecule and at least two binding molecules specific for the target molecule, wherein a first of the binding molecules is associated with a first label and a second of the binding molecules is associated with a second label, wherein a particle having the first label and a particle having the second label are distinguishable from a particle having both the first and second labels, wherein the first and second binding molecules each comprise at least two binding regions specific for the target molecule. The present invention also discloses a composition comprising a first binding molecule associated with a first label and a second binding molecule associated with a second label, wherein the signal obtained from the first and second labels is distinguishable from the combined signal of the first and second labels, characterized in that the first and second binding molecules each comprise at least two binding regions specific for essentially the same target molecule, preferably for essentially the same epitope on the target molecule and a method for selecting a synthetic antigen from a collection of at least three antigens comprising using the disclosed composition and method. The invention further discloses an antigen obtainable by the above-described method and capable of inducing an early immune response, a kit of parts to perform the method, the use of antigens selected by the methods for use as a vaccine, and the use of antibodies and antigens as a medicament.

### CROSS-REFERENCE TO RELATED APPLICATIONS

This application is a continuation of PCT International Patent Application No. PCT/NL2005/000101, filed on Feb. 11, 2005, designating the United States of America, and published, in English, as PCT International Publication No. WO 2005/078450 A2 on Aug. 25, 2005, which application claims priority to European Patent Application No. 04075439.2, filed on Feb. 12, 2004, the contents of the entirety of each of which are hereby incorporated herein by this reference.

### TECHNICAL FIELD

The invention relates to the field of immunology and vaccine development. The invention further relates to the detection of early B-cell responses after immunization with specific antigens.

### BACKGROUND

Vaccines have proven to be extraordinary effective means to improve public health (Jackson et al., 2002). However, conventional vaccine development can be hampered by technical problems in pathogen inactivation, as well as high costs.

Furthermore, many antigens are not readily recognized by the immune system as foreign and consequently, they elicit no adequate immune response. In these cases, peptide-based synthetic vaccines are valid alternatives (Jackson et al., 2002). Peptide vaccination has been successfully used to prevent infectious diseases (Monzavi-Karbassi et al., 2002), and to treat tumors (Ribas et al., 2003; Noguchi et al., 2003), amyloidosis (Nicoll et al., 2003) and autoimmune disease (Liu et al., 2002).

One difficulty in preparing vaccines against antigens that are not recognized by the body as foreign is caused by the phenomenon of tolerance. Normally, an immune response is elicited against antigens that are recognized as foreign, but not against self-antigens. In order for a vaccine to be successful, it must be sufficiently foreign. Only when a vaccine is foreign enough, i.e., sufficiently immunogenic, will the immune system respond to the vaccine and an immune response is induced. Conversely, however, the immune cells and/or antibodies must still be capable of recognizing the self-substance or the tolerated antigen and, thus, the vaccine cannot be too “foreign.” For this reason, peptides of the antigens are made and the peptides are modified to enhance the immunogenicity of the antigens. The peptides or modified peptides are used as a peptide vaccine.

The development of a peptide vaccine is a labor-intensive and time-consuming process. Peptide vaccine candidates must elicit an immune response and cause clonal expansion of specific T cells and/or B cells. The immune response is elicited by epitopes that are recognized by T cells and/or B cells and initiate a proliferative reaction of antigen-specific T and B cells. The proliferative reaction syn.: “clonal expansion” starts within a few hours after the initial contact of an antigen with the immune system, for example, after vaccination. The number of specific immune B cells increases by clonal expansion until high levels of effector cells are reached after three or four weeks after vaccination. The levels of these effector cells after clonal expansion are high enough for conventional methods to detect the effector cells. To detect whether peptides expose B or T cell epitopes, screening of a large set of peptides in animal immunization procedures is necessary. Because the immune repertoire of B-cell and T-cell specificities is very diverse (Davis and Bjorkmann, 1988) and the frequency of antigen-specific B cells participating in an antibody response is very low, detection of B cells presents a major difficulty in its functional and molecular characterization (Newman et al., 2003) and poses a major problem for investigators studying B-cell reactions. After clonal expansion, activated B cells produce antigen-specific antibodies (Adams et al., 2003). Therefore, B-cell response against an antigen is usually measured by measuring the level of antigen-specific antibodies in circulating blood. As a result, a large number of experimental animals has to be kept for a considerable amount of time because after normal immunization procedures, it takes at least four to five weeks before antibodies directed against an antigen have reached levels at which they can be compared and interpreted. In effect, this provides a major obstacle for fast and systematic synthetic peptide vaccine development.

B cells can be detected specifically by direct or indirect staining methods, usually achieved by coupling antigens or antibodies to a signaling molecule, like, for example, a fluorescent stain (fluorescein isothyocyanate, FITC, or Rhodamine), or a chemical, radioactive, or enzymatic signal or another marker substance that enables a skilled person to detect binding of the marker substance to a B cell. Many methods have been developed to increase the sensitivity of B-cell detection to a level where small numbers of antigen-specific B cells can be detected in a large collection of cells, like, for example, in spleen cells. Until now, the numbers of antigen-specific B cells before the process of clonal expansion were too low to be detected by existing detection methods. The clonal expansion process increases the number of B cells to a level where either B cells or antibodies produced by the B cells, can be detected. Based on fluorescent staining technology, flow cytometry was developed and further adapted to detect antigen-specific B cells (McHeyzer-Williams et al., 1993). Historically, investigators have employed and combined two different approaches to study antigen-specific B cells. One approach is the hybridoma technique to capture immune reactive B cells and the other approach is to study genetically manipulated animals with an enhanced number of antigen-specific B cells. Both methods are too insensitive to detect the low levels of emerging antigen-specific B cells after vaccination before clonal expansion has been completed.

One method developed for the detection of B cells has been adapted from the staining of T cells. The sensitivity of detecting T cells was enhanced by incubating the cells with a tetrameric peptide-MHC molecule that binds to T cell receptors specific for the peptide-MHC complex (Altman et al., 1996; Stetson et al., 2002). Although the above-mentioned method has been adapted for the detection of antigen-specific B cells (Newman et al., 2003), the sensitivity of the normal procedures was only increased ten-fold. This is still far too insensitive for effective detection of emerging low-frequency antigen-specific B cells shortly after vaccination.

Another method to enhance the detection level of low-frequency target cells has been described by Townsend et al., 2001, who succeeded in lowering the detection threshold by staining B cells with two reagents specific for the same B-cell receptor that had been conjugated to two different labels (dubbed “single epitope multiple staining”). The principle of this sequential staining procedure (in which the first reagent is used at sub-saturating concentrations) is that the probability of false positive cells binding both reagents is much smaller than the probability of false positive cells binding only one of the reagents. However, Townsend et al. only characterized adoptively transferred BCR-transgenic cells, leaving the question unanswered as to whether their methods could be extrapolated to the analysis of bona fide immune responses, including those induced by vaccines.

This “single epitope multiple staining procedure” reduced the background staining, thereby enabling an increase in the sensitivity of flow cytometry by 1 or 2 orders of magnitude enabling the detection of high-affinity antigen-specific B cells in vivo. This increase in sensitivity may enable the detection of transgenic B cells or B cells after clonal expansion is completed, but it is still too insensitive for the detection of low-frequency early B cells within seven days after immunization. Furthermore, the method is complicated and the authors warn the public that it is critical to note that several requirements must be met before this strategy will be effective in increasing the sensitivity of flow cytometry. The above-described method of Townsend et al. used, as an antigen, a foreign antigen for mice, namely, chicken egg white lysozyme. When applied to the detection of specific B cells against a self-antigen like GnRH, the method could not discriminate any specific B cells from the background staining (see Example 1 of the working examples in this application). Therefore, the method of Townsend et al. was not suited for the detection of low-frequency B cells against self-antigens.

### SUMMARY OF THE INVENTION

We have developed a highly sensitive and reproducible affinity-binding assay to select peptide vaccine candidates based on specific staining of emerging B cells during the clonal expansion at a very early stage of the immune response. An affinity-binding assay is a test for detecting or measuring binding of two or more substances that have a certain affinity for each other. This can, for example, be the binding between an antibody and an antigen, or of an enzyme to its substrate, or of a hormone to its hormone receptor. In the present application, we describe, as an example, the affinity binding between the antigen-specific B-cell receptor to an antigen. Of course, the affinity binding may be detected and measured by various methods. As an example, we disclose the detection of affinity binding by an adapted flow cytometry method.

The present application discloses a binding assay and a test method using this binding assay in which a particle or a cell comprising at least four target molecules, for example, membrane-bound antibodies or receptors or antigen-specific B-cell receptors (BCRs), can be detected by contacting the particle or cell with at least two binding molecules that have a different label. Detection of particles or cells by flow cytometry is based on staining the particle or cell with a fluorescent label and detecting labeled particles or cells by registering fluorescence signals using the fluorescence detectors. Each fluorescent label has its own characteristics for intensity and the level of background staining. This application discloses that staining with a molecule comprising a multiple form of an antigen, in which at least two, or three, preferably four, or more, binding epitopes of essentially the same antigen are present and exposed, increases the intensity of the staining. A further increase in sensitivity is achieved by combining the staining of the above-described multiple forms of an antigen with two different fluorescent labels. For this purpose, two quantities of essentially the same multiple antigen molecules are associated with two different labels. In this case, labels are selected that differ in their fluorescent signal, like, for example, R-phycoerythrin (PE)-labeled neutravidin and allophycocyanin (APC) coupled to streptavidin. By contacting the cells with both differently labeled antigenic molecules, the cells bind both molecules and combine the two different fluorescent labels on the surface. This contacting can, for example, be done in consecutive steps, for example, by first incubating the cells with the first fluorescent-labeled antigen molecule in a sub-saturating amount, followed by incubation with the second fluorescent-labeled antigen in a more saturating or a saturated amount. Another example of a way of staining particles or cells with both fluorescent-labeled antigen molecules is by mixing the two differently labeled antigen molecules at an approximately equimolar amount and incubating the particles or cells with the mixture. Because the chance of aspecific binding of two separate molecules on the same particle is very small, the specificity of the method is increased. This increased specificity, combined by the higher avidity of the binding with multiple antigen molecules, causes a surprising increase in sensitivity and allows for the specific detection of only 20 to 30 cells in one million cells. In this application, the antibody molecules or the antigen-specific B-cell receptors present on the surface of the immune cells are called the target molecules or a member of an affinity binding pair, the member being specific for an epitope in a proteinaceous molecule. The antigen molecules that bind to the target molecules are in this application named binding molecules or molecules. The (binding) molecules may comprise one or more peptides or proteins or fragments thereof. In addition, the (binding) molecules may comprise a staining molecule. A particle in this application can be a virus or a microorganism, or a part of a cell or yeast. Preferably, the size of the particle is at least about the size of a virus and at most about the size of a thousand cells, more preferably about the size of a hundred cells, even more preferably about the size of ten cells, even more preferably about the size of one cell. In this patent application, we use the phrase “distinguishable” for the fact that by interpreting the results of preferably flow cytometry, double-stained particles or cells can be distinguished from particles or cells that have only one label or no label at all. Preferably, this distinction is made through distinction of the signal from the first and second labels. “Label” means in this context preferably a fluorescent-staining label, but may also be a magnetic label or any other kind of staining label.

Therefore, the present invention discloses an affinity-binding assay comprising a particle having at least two copies of a target molecule and at least two binding molecules specific for the target molecule, wherein a first of the binding molecules is associated with a first label and a second of the binding molecules is associated with a second label, wherein a particle having the first label and a particle having the second label are distinguishable from a particle having both the first and second labels, wherein the first and second binding molecules each comprise at least two binding regions specific for the target molecule. The particle can also be a cell, therefore, the invention also discloses the above-described assay wherein the particle comprises a cell. The cell may be a living cell or the cell may be treated with a fixative such as, for example, fixatives that are normally used in flow cytometry, like, for example, formalin, acetone, alcohol, or glutaraldehyde.

Because the method of the invention is very sensitive, this is the first time that such a method is capable of detecting very low frequency antigen-specific B cells, like, for example, very early immune B cells. These very early immune cells are only present at very low frequency because these cells have not yet accomplished the complete clonal expansion process. Therefore, the present invention discloses an affinity-binding assay as described above wherein the cell comprises an activated immune cell in the clonal expansion phase of a primary immune response. In mice, for example, this activation takes place within hours after first or second contact with an antigen and results in antibody levels in peripheral blood within three to four weeks.

The method is especially suitable for the detection of very low-frequency B cells and saves a lot of time because one does not have to wait any longer for the antibody response to evolve. This shortens the animal phase for each peptide from at least three to four weeks to one or two weeks. Furthermore, the detection of very low-frequency B cells is also very suitable for the detection of memory B cells. This may be important for assessing the immune status against certain diseases. A B cell in this application means a B cell in any state of activation or differentiation, including, for example, antigen-specific precursor B cells, antibody-secreting cells, plasma cells and memory B cells. Therefore, the present invention discloses in a preferred embodiment the assay as described above wherein the cell comprises a B cell. The possibilities to count and phenotypically characterize B cells as disclosed in this application enable a person skilled in the art to measure the early immune response against immunization or infection, e.g., proliferation and differentiation of naïve B cells into antibody-secreting cells, memory B cells and plasma cells.

Because the combination of the multiple-antigen molecules with the multiple-staining technique increases the sensitivity at a surprising amount, the present invention discloses an affinity-binding assay as described above, wherein the particle having the first and second labels increases the sensitivity of the affinity-binding assay.

The binding properties of the multiple antigen molecules are increased with the number of exposed antigenic sites on the molecule. In this application, tetramer molecules comprising four antigenic sites have been tested as an example. Of course, other numbers of antigens repeated in the same molecule may also have the desired effect. Therefore, the present invention discloses an affinity-binding assay as described above wherein the first and second binding molecules each comprise at least two binding regions specific for the target molecule. Preferably, the number of binding regions is four.

The binding may be increased even further if the antigenic sites or epitopes of the target molecule are essentially identical epitopes. Therefore, the present invention discloses an affinity-binding assay as described above wherein the at least two binding regions are essentially identical. A big advantage of the assay is that the binding molecules of the detection assay may represent an epitope of a native protein, because in this way, immunization with altered antigens of the native protein can be monitored for the cross-specificity for the native protein. A native protein is in this application is a protein as it is present in nature. Therefore, the present invention discloses an affinity-binding assay as described above wherein the binding molecule represents an epitope of a native protein. The present invention is very suited for detecting low-frequency B cells. The B cells, when activated by contact with an antigen, expose during their clonal expansion an antigen-specific B-cell receptor, or BCR, on their cell surface. Suitable B-cell receptors are, for example, the well-known cell-bound antibody-like molecules that are inserted in the wall of the B cell with their Fc fragment and that are specifically recognizing antigen with the variable region, the antigen being the antigen that was used to activate the B cells (Kouskoff et al., 2000). In the described assay, the BCRs are the target molecules with which the binding molecules bind. Therefore, the present invention discloses an affinity-binding assay as described above wherein the target molecule comprises a BCR.

Even more preferably, the assay detects binding to the antigen-specific site of the BCR. Therefore, the present invention discloses an affinity-binding assay as described above wherein the target molecule comprises a variable region of the BCR.

The assay is performed with a set of binding molecules detecting the target molecule, the binding molecule preferably comprising tetramer molecules. Therefore, the present invention in another embodiment discloses a composition comprising a first multiple-binding molecule associated with a first label and a second multiple-binding molecule associated with a second label, wherein the signal obtained from the first label and the second label is distinguishable from the combined signal of the first and second labels, wherein each binding molecule comprises at least four binding regions specific for essentially the same target molecule, preferably for essentially the same epitope on the target molecule.

Now that there is an assay and a composition for detecting and selecting B cells at a very early stage during clonal expansion, there is no longer a need to wait for at least three to four weeks for antibody levels before an antigen can be evaluated for vaccine purposes. This enables a person skilled in the art to select, in a fast and reliable way, an antigen from a large collection of antigens. In another embodiment relating to therapeutic vaccination procedures involving, for instance, tumor antigens and hormones, individual responses can be monitored and non-responsive individuals can be identified more rapidly.

To overcome the tolerance of the immune system for certain antigens, a range of modified antigens can be produced, for example, by the production of synthetic peptides. Changes in the peptides may increase the immunogenicity, and by testing the peptides, new and improved antigens may be detected and selected by the assay and the method of the invention. A range of changes made to a peptide in order to change the antigenicity of the peptide, results in various forms of the peptide, which is also called a collection of peptides. Antigen-specific B-cell detection as disclosed in this application expedites the evaluation of vaccine immunogenicity and enables analysis of the fine-specificity of the response. For instance, the precise (sub)-serotype specificities of the novel generation of multivalent conjugate vaccines can now be analyzed in unprecedented detail. Therefore, the present invention discloses a method for selecting a synthetic antigen from a collection of at least two antigens comprising using the composition as described above for detecting in an affinity-binding assay for immune cells specific for the synthetic antigen, clonal expansion of antigen-specific immune cells in samples of cells obtained from a mammal immunized with an antigen of the collection of antigens, and comparing the clonal expansion with the clonal expansion of another mammal immunized with another antigen from the collection and selecting from the collection an antigen with which clonal expansion was more extensive than clonal expansion observed with at least one other antigen from the collection. A “more extensive clonal expansion” means in this application that the numbers of specific B cells increase faster and to higher levels, compared to clonal expansions as a reaction to other antigens. In mice, for example, increased clonal expansion at seven days is indicative for an increased clonal expansion at 14 days and usually for a high level of antibodies later in the immune response. In rats or other animals, this increase may show later or earlier in the immune response but be highly indicative of a more extensive clonal expansion, if compared to the clonal expansion in other rats or other animals immunized with another antigen of the collection of antigens.

Of course, the above-described method may use any of the affinity-binding assays as are described above. Therefore, the present invention discloses a method for selecting a synthetic antigen as described above wherein the affinity-binding assay comprises any assay as described herein.

Of course, for the synthetic peptides to be a good vaccine against a native protein, cross-reactivity of the antibodies directed against the synthetic peptide antigen with the native protein from which the peptides were derived is highly appreciated. Therefore, the method as described above is preferably used wherein the at least two binding molecules comprise as a binding part for the target molecule a synthetic antigen of the collection of antigens, or a native antigen or homologue of the antigen. A “native antigen” is, in this application, an antigen as it occurs in a protein in nature. A homologue of an antigen is a substance showing the same antigenic characteristics in kind, but not necessarily in amount. The homologue may comprise a natural peptide or protein or a synthetic peptide or a part thereof. Therefore, direct B-cell staining procedures enable the assessment of specific B-cell receptor cross-reactivity between peptide vaccines and the native antigens from which they are derived.

The invention also discloses the method as described above, further comprising selecting an antigen for which clonal expansion of antigen-specific immune cells is more extensive than the clonal expansion with at least two other antigens.

With the assay and the method as described above, B cells specific for a certain antigen can be selected and isolated. The isolated B cells may also be further tested for the specificity and avidity of their binding to the native protein, also called the native antigen or homologue of the antigen. Therefore, the invention teaches a method as described above further comprising evaluating whether the antigen-specific immune cells are specific for a native antigen or homologue of the antigen.

The above-described methods and assays enable a person skilled in the art to select and isolate antigen-specific cells. Therefore, the invention discloses a method for an affinity-binding assay for selecting antigen-specific immune cells, comprising: (a) contacting a cell having at least four copies of a target molecule with at least two binding molecules, preferably tetrameric binding molecules, the binding molecules specific for the target molecule wherein a first of the binding molecules is associated with a first label and a second of the binding molecules is associated with a second label; (b) detecting cells staining with each label; and (c) selecting cells binding both labels.

The present invention teaches with the above-described methods a new way to test and select a synthetic antigen from a collection of antigens inducing a cross-reactive immune response with a native protein. This is an important improvement of the technical possibilities in this field and opens the possibility for new methods of selection and thus to other products. Therefore, the invention discloses a synthetic antigen, inducing a cross-reactive immune response with a native protein, selectable with a method as described above for use as a vaccine. Of course, isolated peptides, derived from a proteinaceous molecule, are capable of inducing an immune response to a proteinaceous molecule.

To increase the antigenicity of a peptide, the peptide motif can be made into dimeric, trimeric or multimeric peptides wherein two or three or more peptides are linked directly to each other or through a spacer molecule. Combining two identical peptides into a dimer peptide further increases the immunogenic properties of the peptide. Therefore, the present invention discloses an immunogenic peptide, obtainable by the above-described method, wherein the peptide comprises a dimer peptide.

In the dimer peptide, the peptides can be linked by sulphur bridges or by a linkage molecule. Another method of increasing the immunogenicity of a peptide is by combining a peptide in a tandem peptide and/or a tandem-dimer peptide. From WO 96/40755 it is known that the tandem-dimer principle applied to a variant of the GnRH-I molecule resulted in a vaccine that was highly effective in low doses and with a mild adjuvant. Therefore, the present application discloses a method for selecting an immunogenic peptide derived from an antigen comprising detecting clonal expansion of antigen-specific immune cells as described above wherein the peptide comprises a tandem-dimer peptide.

The antigenic properties of peptides provided herein are further optimized using a variety of techniques, such as replacement-net mapping, allowing detecting peptides with improved characteristics. For example, one such peptide may be a peptide that binds to an antibody directed against an epitope, thereby mimicking the immunogenic properties of the epitope. Such a peptide is called a mimotope. This method has been disclosed in patent application WO 00/29851. Therefore, the present application also discloses an immunogenic peptide derived from a protein comprising a mimotope of the protein.

Amino acid substitution of at least one amino acid by a D-amino acid and/or a L-alanine as described in WO 02/22659 also increases the immunogenicity of a peptide. Therefore, the present invention discloses an immunogenic peptide derived from a protein wherein at least one amino acid is substituted by a D-amino acid and/or a L-alanine.

One example of a peptide hormone for which peptides have been prepared according to the sequence of the hormone and which are tested in the above-mentioned method is gonadotrophin-releasing hormone or GnRH. Therefore, the application discloses the above-mentioned methods wherein the antigen comprises a GnRH-peptide hormone and/or part thereof or a GnRH-derived peptide. Gonadotrophin-releasing hormone (GnRH) is a decapeptide, produced by the hypothalamus. The GnRH travels through portal circulation to the pituitary to stimulate the release of gonadotrophins FSH and LH (Talwar, 1999). Anti-GnRH immunization blocks the fertility of both male and female animals (Meloen et al., 2001), and GnRH vaccines have applications in prostate and female cancers (Talwar, 1999; Fuerst et al., 1997). To compare the usefulness of the different methods, mice were immunized with GnRH-like peptides conjugated to ovalbumin (OVA). Three different peptides were produced, i.e., GnRH-monomer (pEHWSYGLRPGC (SEQ ID NO:1)) GnRH-tandem (pEHWSYGLRPGQHWSYGLRPGC (SEQ ID NO:2)) and GnRH-tandem dimer (TDK, the dimerized form of pEHWSYkLRPGQHWSYkLRPGC in which “k” represents a D-lysine residue). The GnRH tandem and TDK peptide conjugated to a carrier protein have been shown to be highly immunogenic, resulting in GnRH-neutralizing antibodies and a full biological effect in all treated animals (Meloen et al., 1994; Oonk et al., 1998). Therefore, the present application provides an immunogenic peptide obtainable by the above-described method wherein the peptide is derived from GnRH.

Another example of a peptide hormone for which peptides have been prepared according to the sequence of the protein and which are tested in the above-mentioned method is Gastrin. Therefore, the application discloses an immunogenic peptide derived from a peptide hormone and obtainable by the above-mentioned methods wherein the peptide is derived from Gastrin. Gastrin is a peptide hormone that is important in the regulation of acid secretion and the growth of both normal and malignant gastrointestinal epithelium (Jensen et al., 2002). Tumor cells can respond both to circulating endocrine gastrin (Watson et al., 1989) and locally produced gastrin, which acts in an autocrine or paracrine manner (Hoosein et al., 1990). Effective inhibition of gastrin may be beneficial as a therapy for colorectal cancer, gastric cancer and pancreatic cancer, as gastrin acts as a growth factor for these tumors. Gastrin receptor antagonists are not fully effective because several receptor subtypes are involved in the action of gastrin and high amounts are required to displace gastrin. Neutralization of gastrin may circumvent this problem.

Peptides derived from gastrin can be used for vaccination against gastrin (amino acid sequence pEGPWLEEEEEAYGWMDF (SEQ ID NO:3)). Suitable peptides for vaccination include amino acid 1-X wherein X=5 to 12. A Cysteine is included at the N- or C-terminal end for conjugation purposes. The peptides can also be applied in tandem or tandem dimer formulation with a Cysteine added at the N- or C-terminus or a cysteine between the two gastrin sequences in the tandem, as in pEGPWLEEEECQGPWLEEEE (SEQ ID NO:4). L-Lysines or D-Lysines are introduced in the tandem formulation to allow dimerization of these peptides via the cysteine and conjugation via the L- or D-Lysine, like in

| | |
|---|---|
| pEGPWLEEEEQGPWLEEEECK# | (see, SEQ ID NO:5) |
| \| | |
| EGPWLEEEEQGPWLEEEECK# | |
| pEGPWLEEEEEAYkQGPWLEEEEEAYkC# | (see, SEQ ID NO:5) |
| \| | |
| pEGPWLEEEEEAYkQGPWLEEEEEAYkC# | |

in which “k” represents a D-lysine residue.

These peptides were tested as a vaccine in mice and at seven and 14 days after immunization, specific B cells were detected with the method of the invention using tetramer Gastrin peptide molecules. The following peptide was used to form a tetramer: pEGPWLEEEEEAYGWMDFK($)# (SEQ ID NO:6), wherein # is amide; pE is pyroGlutamine; and K($) is biotinylated Lysine.

Therefore, the present invention discloses an immunogenic peptide obtainable by any of the above-described methods wherein the peptide is derived from Gastrin.

Yet another example of a hormone for which peptides have been prepared is Human Chorionic Gonadotropin-beta (hCG-β), which is normally produced by the placenta during pregnancy (Meyer et al., 2001). The protein can be detected in serum or urine. hCG-beta is elevated during the first trimester of pregnancy and is often used as an indicator of pregnancy. hCG-β is also a tumor-associated antigen that is in various types of cancer. Most commonly, hCG-beta is elevated, >10 mIU/ml (Gauchi et al., 1981), in gynecological cancers (Gauchi et al., 1981; Higashida et al., 2001; Kinugasa et al., 1992), but also in colorectal (Lundin et al., 2000), seminoma testicular (Hara et al., 2002), bladder (Hotakainen et al., 2002), liver (Szavay et al., 2000), stomach (Bateman et al., 1995), pancreas (Syrigos et al., 1998), lung (Uckaya et al., 1998), brain (Fujimaki et al., 2000), and kidney (Hotakainen et al., 2002) cancers. Non-malignant elevations may be observed in pregnancy (Meyer et al., 2001), ulcers (Manabe et al., 1985), duke's disease (Carpelan-Holmstrom et al., 1996), and cirrhosis (Hoermann et al., 1992). Levels of hCG-β are useful in monitoring the effectiveness of treatment, such as chemotherapy, against tumor progression. Cancer patients with elevated levels of hCG-β have significantly shorter survival time than patients with levels below the median level. Effective inhibition of hCG-β is, therefore, beneficial as a therapy in gynecological, colorectal, seminoma testicular, bladder, liver, stomach, pancreas, lung, brain, and kidney cancers, both in the prevention of the disease and in advanced disease. Effective inhibition of hCG-β is, therefore, also beneficial as a therapy in ulcers, duke's disease, and cirrhosis. The accession number of the hCG-β sequence is: P01233. The Swiss-Protein entry name is: CGHB_HUMAN. The hormone comprises 165 amino acids wherein amino acids 1-20 form a signal peptide.

Overlapping 20-mer peptides were used for immunization with a N-terminal Cysteine for conjugation to a carrier protein. The following peptides were included: 21-40, 31-50, 41-60, 51-70, 61-80, 71-90, 81-100, 91-110, 101-120, 111-130, 121-140, 129-165 and 141-165. In example 4, we have used a C-terminal peptide (CTP) comprising a sequence corresponding to amino acid position 109-145 of hCG.

Yet another hormone for which peptides have been prepared is Human Parathyroid hormone-related protein (PTHrP).

Hypercalcemia is one of the most common metabolic complications in patients with cancer. Hypercalcemia occurs in a majority of the epithelial cancers in an advanced stage and is caused by PTHrP secretion by the tumor. PTHrP levels are undetectable in healthy persons. PTHrP secreted by tumors causes high calcium levels because it acts on the common PTH receptor.

PTHrP plays an important role in breast cancer. Approximately 50% of the primary tumors secrete PTHrP. This feature is associated with development of bone metastases. In these metastases, PTHrP induces osteolytic bone lesions and is responsible for destruction of bone tissue. This osteolytic bone resorption results in release of TGF-B from the bone tissue, which in turn stimulates the production of more PTHrP.

There are two types of hypercalcemia in cancer:

Humoral hypercalcemia, wherein primary tumors are producing PTHrP, which acts as a hormone, and after being secreted into the bloodstream, it acts on bone and kidney to increase calcium levels.

Local osteolytic hypercalcemia: PTHrP also plays a role in bone metastases. It can induce a local osteolysis near a bone metastasis, allowing the tumor metastasis to grow into the bone tissue. In breast cancer, almost all metastases contain high levels of PTHrP whereas only 17% of the non-bone metastases expresses PTHrP. It causes fractures and bone-pain in these patients. The current treatment for hypercalcemia is administration of bisphosphonates, which reduces calcium levels. But this treatment does not affect the PTHrP levels.

Neutralization of PTHrP in cancers by vaccination against PTHrP may contribute to an improved quality of life of patients with advanced cancer regression. The Swiss-Prot entry name is: PTHR_HUMAN. The accession number of the sequence is P12272. The length of human PTHrP is 177, amino acids 1-177, wherein amino acids 1-36 comprise a leader sequence.

Effective peptides are N-terminal peptides of the PTHrP fragment 37-70, including 37-X, wherein X=47 to 56. A C-terminal Cysteine is added for conjugation. The peptides can be used as tandems and tandem dimers.

The present invention also discloses the use of an immunogenic protein and/or part thereof and/or an immunogenic peptide, selectable with a method as described above for the preparation of a medicament.

In another embodiment of the invention, specific emerging B cells can be sorted and isolated by, for example, cell sorting and/or magnetic bead separation.

The isolated B cells can be immortalized according to methods generally known in the field of hybridoma production and thus become immortalized B cells producing monoclonal antibodies. Therefore, the present invention also discloses a B cell selected with the method as described above for the preparation of an antibody, and the use of such a B cell for the production of a hybridoma.

Monoclonal antibodies produced by the B-cell hybridoma are particularly useful for diagnostic purposes or for the preparation of a medicament, for example, for the inactivation of GnRH in the circulation or for the specific destruction of a tumor cell, or for inhibiting the replication of a pathogen. Therefore, the present invention also discloses an isolated antibody produced by the above-described B cell and the use of the antibody for the preparation of a medicament.

### DESCRIPTION OF THE FIGURES

FIG. 1: Visualization of antigen-specific B cells. Several flow cytometry strategies have been developed to detect specific B cells and we have compared their effectiveness.

FIG. 1A: Staining with a single fluorescent-labeled peptide. Splenocytes from immunized and non-immunized mice stained with C-biotinylated GnRH and FITC-labeled anti-CD19, followed by staining with APC-labeled streptavidin.

FIG. 1B: Splenocytes from immunized (I) and non-immunized mice (N) were stained with C-biotinylated and FITC-labeled GnRH, followed by staining with APC-labeled streptavidin (according to Townsend et al., 2001).

FIG. 1C: Staining with a single fluorescent-labeled tetramer (according to Newman et al., 2003). Splenocytes from immunized (I) and non-immunized mice (N) were stained with the PE-labeled GnRH tetramer and FITC-labeled anti-CD19.

FIG. 1D: Staining with two fluorescent-labeled tetramers, using the single epitope multiple-staining principle of this application. Splenocytes from immunized (I) and non-immunized mice (N) were stained with the PE-labeled and APC-labeled GnRH tetramers and with FITC-labeled anti-CD19.

FIG. 2: Identification of GnRH and of Gastrin-specific B cells.

FIG. 2A: Specific B cell-tetramer double-staining. Splenocytes from mice immunized with GnRH-TDK or Gastrin-TDK were stained with peptide-tetramer molecules, anti-mouse CD3e, anti-mouse CD19 and analyzed by FACS. Gating strategy for identification of tetramer-reactive lymphocytes includes a live gate (forward and side scatter) and a negative gating on CD3e for excluding T cells. Using two different peptide-tetramer molecules, linked with PE or APC, we were able to detect specific GnRH cells at seven and 14 days after immunization. All Dot Plots derived from a minimum of three independent experiments in which at least five experimental and five control mice were analyzed.

FIG. 2B: Specific tetramer double-positive cells are CD19 positive. After the identification of the tetramer double-positive cells, we made a gate (R3) to visualize these cells alone. Then, we looked at the phenotypic expression of CD19 on these cells. All the specific cells are CD19 positive. The density plots are representative of all the lymphocytes population and they were made to select the right size of the quadrants for the discrimination of the CD19-positive cells.

FIG. 3: Specificity of the B-cell detection.

FIG. 3A: Unlabeled GnRH or Gastrin is able to inhibit the peptide-tetrameric molecule staining of the specific B cells. The specificity of tetramer binding was demonstrated by inhibition studies. Splenocytes were pre-incubated with unlabeled GnRH or Gastrin, followed by incubation with labeled tetramers and anti-CD19 and anti-CD3 antibodies. Three doses of unlabeled antigens were used: 2 mM, 20 μM and 0.2 μM. Tetramer staining was nearly abolished by the higher dose.

FIG. 3B: Specific B cells are recognized only by C-term tetramer. Peptide-tetrameric molecules made with different biotinylated GnRH: C-term, N-term, and middle biotinylated peptides. Only the C-term peptide was able to detect the specific cells.

FIG. 4: Detection of antigen-specific B cells by ELIspot assay and Elisa test.

FIG. 4A: ELIspot assay. After lysing the erythrocytes, splenocytes from non-immunized mice and from mice 14 days after immunization were cultivated for five days under Pansorbin and IL-2 stimulation. After this incubation, cells were added to ELIspot wells coated with peptide-tetramer, diphtheria toxoid and anti-mouse IgG. On the wells coated with anti-mouse IgG, it is possible to evaluate the total B-cell response of the spleens. On the others, it is possible to evaluate the specific B-cell response against the antigen. Only cells from immunized mice were able to produce spots on the specific antigen.

FIG. 4B: Elisa test. The supernatants collected from the cells after five days of culture are used to perform an Elisa test. The level of total B-cell response is identical between immunized and non-immunized mice. Only sera from immunized mice contain antibodies against gastrin and diphtheria toxoid. The levels of specific antibodies are higher 14 days after immunization than after seven. These results are consistent with the FACS analysis.

FIG. 5: Counting GnRH-specific B cells after immunization with the three GnRH vaccines.

FIG. 5A: Specific B-cell tetramer double-staining. Splenocytes from mice immunized with GnRH-mono, GnRH-tandem, and TDK-tandem dimer were stained with GnRH-tetramers (PE and APC), PerCP labeled anti-CD3, FITC-labeled anti-CD19 and analyzed by flow cytometry. Tetramer-binding lymphocytes were identified using a live gate (forward and side scatter) and a negative gate on FL3+ to exclude (irrelevant) T cells and autofluorescent cells. For each density plot, 1×10⁶ events were acquired. Shown in the upper right panel are the double-positive-specific B cells. Plots shown are representative from a minimum of three independent experiments in which at least three experimental and three control mice were analyzed.

FIG. 5B: For each vaccine, at least nine samples were analyzed seven days and 14 days after immunization. Shown in the graph is the mean of the number of double-positive cells found in each group.

FIG. 6: Counting Gastrin-specific B cells after immunization with the four Gastrin vaccines.

FIG. 6A: Specific B-cell tetramer double-staining. Splenocytes from mice immunized with Gastrin-mono, Gastrin-tandem, TDK1-tandem dimer and TDK2-tandem dimer were stained with Gastrin-tetramers (PE and APC), PerCP-labeled anti-CD3, FITC-labeled anti-CD19 and analyzed by flow cytometry. Tetramer-binding lymphocytes were identified using a live gate (forward and side scatter) and a negative gate on FL3+ to exclude (irrelevant) T cells and autofluorescent cells. For each density plot, 1×10⁶ events were acquired. Shown in the upper right panel are the double-positive-specific B cells. Plots shown are representative from a minimum of three independent experiments in which at least three experimental and three control mice were analyzed.

FIG. 6B: For each vaccine, at least nine samples were analyzed seven days and 14 days after immunization. Shown in the graph is the mean of the number of double-positive cells found in each group.

### DETAILED DESCRIPTION OF THE INVENTION

The techniques described in this patent application may be used for selecting suitable antigens for inducing an immune response. Once an antigen is known, the present application shows how to increase the labeling sensitivity by making binding molecules comprising multiple copies of the antigen, how to label the binding molecules, and how to use the labeled binding molecules for the selection of an antigen. Therefore, this application discloses a kit of parts comprising at least a first binding molecule associated with a first label and a second binding molecule associated with a second label, the binding molecules comprising at least two binding regions for the same target molecule. A kit as above described is very useful to select a peptide from a library of peptides. Therefore, the present invention discloses the use of the kit for the selection of an antigen.

The invention provides an affinity-binding assay comprising a particle having at least four copies of a target molecule and at least two binding molecules specific for the target molecule, wherein a first of the binding molecules is associated with a first label and a second of the binding molecules is associated with a second label, wherein a particle having the first label and a particle having the second label are distinguishable from a particle having both the first and second labels, wherein the first and second binding molecules each comprise at least two binding regions specific for the target molecule.

The invention further discloses an affinity-binding assay of the invention wherein the particle comprises a cell.

The invention further discloses an affinity-binding assay of the invention wherein the cell comprises an activated immune cell in the clonal expansion phase of a primary immune response.

The invention further discloses an affinity-binding assay of the invention wherein the cell comprises a B cell.

The invention further discloses an affinity-binding assay of the invention wherein the particle having the first and second labels increases the sensitivity of the affinity-binding assay.

The invention further discloses an affinity-binding assay of the invention wherein the first and second binding molecules each comprise at least four binding regions specific for the target molecule.

The invention further discloses an affinity-binding assay of the invention wherein at least four binding regions are essentially identical.

The invention further discloses an affinity-binding assay of the invention wherein the binding molecule represents an epitope of a native protein.

The invention further discloses an affinity-binding assay of the invention wherein the target molecule comprises a B-cell receptor.

The invention further discloses an affinity-binding assay of the invention wherein the target molecule comprises a variable region of the B-cell receptor.

The invention further discloses a composition comprising a first multiple binding molecule associated with a first label and a second multiple binding molecule associated with a second label, wherein the signal obtained from the first label and the second label is distinguishable from the combined signal of the first and second labels, wherein each binding molecule comprises at least four binding regions specific for essentially the same target molecule, preferably for essentially the same epitope on the target molecule.

The invention further discloses a method for selecting a synthetic antigen from a collection of at least two antigens comprising using the composition of the invention for detecting in an affinity-binding assay for immune cells specific for the synthetic antigen, clonal expansion of antigen-specific immune cells in samples of cells obtained from a mammal immunized with an antigen of the collection of antigens, and comparing the clonal expansion with the clonal expansion of another mammal immunized with another antigen of the collection, and selecting from the collection an antigen with which clonal expansion was more extensive than clonal expansion observed with at least one other antigen from the collection, wherein the collection of antigens comprises any antigen of the invention.

The invention further discloses a method of the invention wherein the affinity-binding assay comprises an assay of the invention or a method of the invention.

The invention further discloses a method of the invention wherein at least two binding molecules comprise as a binding part for the target molecule, a synthetic antigen of the collection of antigens, or a native antigen or homologue of the antigen.

The invention further discloses a method of the invention, further comprising selecting antigen for which clonal expansion of antigen-specific immune cells is more extensive than the clonal expansion with at least two other antigens.

The invention further discloses a method of the invention, further comprising evaluating whether the antigen-specific immune cells are specific for a native antigen or homologue of the antigen.

The invention further discloses a method for an affinity-binding assay for selecting antigen-specific immune cells, comprising:
contacting a cell having at least four copies of a target molecule with at least two binding molecules specific for the target molecule, wherein a first of the binding molecules is associated with a first label and a second of the binding molecules is associated with a second label;
detecting cells staining with each label; and
selecting cells binding both labels.

The invention further discloses a synthetic antigen, inducing a cross-reactive immune response with a native protein, selectable with a method of the invention for use as a vaccine.

The invention further discloses an antigen of the invention wherein the antigen comprises a dimer peptide.

The invention further discloses an antigen of the invention wherein the antigen comprises a tandem-dimer peptide.

The invention further discloses an antigen of the invention comprising a mimotope of the antigen.

The invention further discloses an antigen of the invention wherein at least one amino acid is substituted by a D-amino acid and/or a L-alanine.

The invention further discloses an antigen of the invention wherein the peptide is derived from GnRH.

The invention further discloses an antigen of the invention wherein the peptide is derived from Gastrin.

The invention further discloses an antigen of the invention wherein the peptide is derived from hCG.

The invention further discloses an antigen of the invention wherein the peptide is derived from PTHrP.

The invention further discloses the use of an antigen of the invention for the preparation of a medicament.

The invention further discloses the use of an antigen of the invention for the preparation of a medicament for the treatment of prostate cancer and of female GnRH-related cancer.

The invention further discloses the use of an antigen of the invention for the preparation of a medicament for blocking fertility.

The invention further discloses the use of an antigen of the invention for the preparation of a medicament for the treatment of Gastrin-related colorectal cancer and/or gastric cancer.

The invention further discloses the use of an antigen of the invention for the preparation of a medicament for the treatment of hCG-related gynecological, colorectal, seminoma testicular, bladder, liver, stomach, pancreas, lung, brain, and kidney cancers.

The invention further discloses the use of an antigen of the invention for the preparation of a medicament for the treatment of hCG-related ulcers, duke's disease, and cirrhosis.

The invention further discloses the use of an antigen of the invention for the preparation of a medicament for the treatment of PTHrP-related cancer.

The invention further discloses the use of an antigen of the invention for the preparation of a medicament for the treatment of PTHrP-related osteolytic bone disease.

The invention further discloses the use of an antigen of the invention for the preparation of a medicament for the treatment of PTHrP-related hypercalcemia.

The invention further discloses an isolated B cell detected with the method of the invention for the preparation of an antibody.

The invention further discloses a hybridoma based on the B cell of the invention.

The invention further discloses an isolated antibody produced by a B cell of the invention or a hybridoma of the invention.

The invention further discloses an isolated antibody of the invention for the preparation of a medicament.

The invention further discloses a kit of parts comprising at least a first binding molecule associated with a first label and a second binding molecule associated with a second label, the binding molecules comprising at least two binding regions for the same target molecule.

The invention further discloses the use of a kit of parts of the invention for the selection of an antigen.

The invention further discloses a method for detecting a particle having at least four copies of a target molecule comprising contacting the particle with a first and second binding molecules having at least two binding sites for the target molecule, the first binding molecule coupled to a first label and the second binding molecule coupled to a second label, and detecting particles having both labels.

The invention further discloses a method for detecting a particle of the invention wherein detecting comprises detection of a combined signal from the first and second binding molecules.

The invention further discloses a method for detecting a particle of the invention wherein the particle is derived from a mammal within 14 days after vaccination with a native antigen or homologue of the antigen.

The invention further discloses a method for detecting a particle of the invention wherein the first binding molecule and the second binding molecule are present at approximately equimolar amounts.

In another embodiment of the invention, the application discloses a method for detecting a cell comprising a member of an affinity binding pair, the member being specific for an epitope in a proteinaceous molecule, the method comprising generating a synthetic peptide derived from at least part of the proteinaceous molecule, the method further comprising immunizing a non-human animal with the synthetic peptide and detecting in a sample obtained from the animal a cell comprising a member of an affinity-binding pair that is specific for the proteinaceous molecule.

In another embodiment, the invention discloses a method of the invention wherein the synthetic peptide is modified such that the modified peptide is immunologically different from any peptide of the proteinaceous molecule, the method further comprising immunizing a non-human animal with the modified peptide and detecting in a sample obtained from the animal, a cell comprising a member of an affinity-binding pair that is specific for the proteinaceous molecule.

In a further embodiment, the invention discloses a method of the invention wherein the sample is incubated with a first molecule comprising at least two copies of the epitope from the proteinaceous molecule or an equivalent thereof comprising a first label, and a second molecule comprising at least two copies of the epitope from the proteinaceous molecule or an equivalent thereof comprising a second label and detecting in the sample a cell that comprises the first and second labels.

In a preferred embodiment, the invention discloses a method of the invention wherein the modified peptide comprises a dimer and/or a tandem-dimer peptide, a mimotope, a replacement-net mapped peptide, and/or an amino acid-substituted peptide comprising between one and four amino acid substitutions per 20 amino acids of the peptide.

In a preferred embodiment, the invention discloses a method of the invention wherein the synthetic and/or modified peptide is derived from GnRH.

In a preferred embodiment, the invention discloses a method of the invention wherein the synthetic and/or modified peptide is derived from Gastrin.

In a preferred embodiment, the invention discloses a method of the invention wherein the synthetic and/or modified peptide is derived from hCG.

In a preferred embodiment, the invention discloses a method of the invention wherein the synthetic and/or modified peptide is derived from PTHrP.

In a preferred embodiment, the invention discloses a method of the invention wherein the cell is an immune cell.

In a more preferred embodiment, the invention discloses a method of the invention wherein the cell is a B cell.

In another embodiment, the invention discloses a method of the invention wherein the first and second molecules comprise tetramers of the epitope or tetramers of the proteinaceous molecule.

In yet another embodiment, the invention discloses a composition comprising a first molecule associated with a first label and a second molecule associated with a second label, wherein the signal obtained from the first label and the second label is distinguishable from a combined signal of the first and second labels, wherein each molecule comprises at least four binding regions specific for essentially the same epitope on a proteinaceous molecule.

In yet another embodiment, the invention discloses a method for selecting an antigen comprising providing a collection of antigens and immunizing at least one non-human animal with the collection and selecting an antigen for which clonal expansion of cells of the B-cell lineage specific for the proteinaceous molecule has occurred.

In a preferred embodiment, the invention discloses a method of the invention wherein the antigen comprises modified peptides derived from at least part of a proteinaceous molecule such that the modified peptides are immunologically different from any peptide of such proteinaceous molecule.

In a preferred embodiment, the invention discloses a method of the invention further comprising selecting an antigen for which clonal expansion of antigen-specific immune cells is more extensive than the clonal expansion with another antigen of the collection.

In a further embodiment, the invention discloses a method of the invention wherein clonal expansion of cells of the B-cell lineage is detected through a method of the invention.

In a preferred embodiment, the invention discloses a synthetic and/or modified peptide capable of inducing a cross-reactive immune response with a native protein obtainable by a method of the invention.

In a more preferred embodiment, the invention discloses a synthetic and/or modified peptide of the invention derived from GnRH.

In a more preferred embodiment, the invention discloses a synthetic and/or modified peptide of the invention derived from Gastrin.

In a more preferred embodiment, the invention discloses a synthetic and/or modified peptide of the invention derived from hCG.

In a more preferred embodiment, the invention discloses a synthetic and/or modified peptide of the invention derived from PTHrP.

In a more preferred embodiment, the invention discloses the use of a synthetic and/or modified peptide of the invention for the preparation of a vaccine.

In a further embodiment, the invention discloses the use of a synthetic and/or modified peptide of the invention for the preparation of a medicament.

In one embodiment, the invention discloses the use of a synthetic and/or modified peptide of the invention for the preparation of a medicament for the treatment of prostate cancer and of female GnRH-related cancer.

In another preferred embodiment, the invention discloses the use of a synthetic and/or modified peptide of the invention for the preparation of a medicament for blocking fertility.

In another preferred embodiment, the invention discloses the use of a synthetic and/or modified peptide of the invention for the preparation of a medicament for the treatment of Gastrin-related colorectal cancer, gastric cancer and/or pancreatic cancer.

In another preferred embodiment, the invention discloses the use of a synthetic and/or modified peptide of the invention for the preparation of a medicament for the treatment of hCG-related gynecological, colorectal, seminoma testicular, bladder, liver, stomach, pancreas, lung, brain, and kidney cancers.

In another preferred embodiment, the invention discloses the use of a synthetic and/or modified peptide of the invention for the preparation of a medicament for the treatment of hCG-related ulcers, duke's disease, and cirrhosis.

In another preferred embodiment, the invention discloses the use of a synthetic and/or modified peptide of the invention for the preparation of a medicament for the treatment of PTHrP-related cancer.

In another preferred embodiment, the invention discloses the use of a synthetic and/or modified peptide of the invention for the preparation of a medicament for the treatment of PTHrP-related osteolytic bone disease.

In a further embodiment, the invention discloses the use of a synthetic and/or modified peptide of the invention for the preparation of a medicament for the treatment of PTHrP-related hypercalcemia.

In another preferred embodiment, the invention discloses a method for determining whether a collection of immune cells comprises a cell having at least two copies of a member of a specific binding pair, specific for an epitope in a proteinaceous molecule, the method comprising:
providing the collection with at least two molecules specific for the member of a specific binding pair, wherein a first of the molecules is associated with a first label and a second of the molecules is associated with a second label; and
determining whether the collection comprises cells stained by both labels.

In a further embodiment, the invention discloses a method of the invention wherein the cell having at least two copies of a member of a specific binding pair, specific for an epitope in a proteinaceous molecule is a B cell.

In another preferred embodiment, the invention discloses a hybridoma cell line derived from the B cell of the invention.

In another preferred embodiment, the invention discloses the use of an isolated B cell of the invention and/or a hybridoma of the invention for the preparation of an antibody.

In a further embodiment, the invention discloses an isolated antibody produced by a B cell of the invention or a hybridoma of the invention.

In a further embodiment, the invention discloses the use of an isolated antibody of the invention for the preparation of a medicament.

In another preferred embodiment, the invention discloses a kit of parts comprising at least a first molecule associated with a first label and a second molecule associated with a second label, the molecules comprising at least two binding regions for the same member of an affinity-binding pair specific for an epitope in a proteinaceous molecule.

In a further embodiment, the invention discloses the use of a kit of parts of the invention for the selection of an antigen.

In a further embodiment, the invention discloses a method for detecting a cell in a collection of cells comprising providing the collection of cells with a first molecule and a second molecule capable of specifically binding the cell, wherein the first and second molecules comprise different labels and wherein the first and second molecules comprise at least four binding sites for the cell, the method further comprising detecting a cell comprising both labels.

In a further embodiment, the invention discloses a method of the invention wherein the cell comprises both labels at approximately equimolar amounts.

In a further embodiment, the invention discloses a method of the invention wherein the cell is an immune cell.

In a further embodiment, the invention discloses a method of the invention wherein the cell is obtained from an animal immunized with a molecule comprising the binding site or a functional derivative thereof.

In a further embodiment, the invention discloses a method of the invention wherein the animal is immunized with a functional derivative that is similar but not identical to a part of a native protein or peptide, and wherein the binding is identical to the part of the native protein or peptide.

In a further embodiment, the invention discloses a method of the invention wherein the functional derivative comprises a dimer peptide.

In a further embodiment, the invention discloses a method of the invention wherein the functional derivative comprises a tandem-dimer peptide.

In a further embodiment, the invention discloses a method of the invention wherein the functional derivative comprises a mimotope of the antigen.

In a further embodiment, the invention discloses a method of the invention wherein the functional derivative comprises from one to six amino acids per 20 amino acids substituted by a D-amino acid and/or a L-alanine.

The invention is further explained in the examples, without being restricted to it.

### EXAMPLE 1

Detection of Low-Frequency Early B Cells.

Materials and Methods

Animals

Six eight-week-old mice were used and were treated according to the ethical guidelines of our institutions. Mice were immunized intraperitoneally on day 0 with GnRH-TDK coupled with ovalbumin (100 μg) or with Gastrin-TDK1 coupled with diphtheria toxoid (Pepscan Systems, Lelystad, NL), in Complete Freunds Adjuvant. Control mice were not immunized. On day 7, half of the mice of each group were boosted, using Incomplete Freunds Adjuvant, and half of the animals were sacrificed and spleens were harvested. On day 14, spleens were harvested from the remaining mice. All animal experiments were done with permission from the local ethical committee (DEC).

Tetramer Synthesis

Tetrameric molecules were produced by mixing equal volumes of C-biotinylated, middle biotinylated or N-biotinylated GnRH at 200 μM (Pepscan Systems) with R-phycoerythrin (PE)-labeled neutravidin at 3.3 μM (PE-NA, Molecular Probes, Eugene, Oreg.), or allophycocyanin (APC)-labeled streptavidin at 6.1 μM (APC-SA, Molecular Probes). Each mixture was incubated at 4° C. for at least 12 hours. Then, tetramers were separated from non-complexed peptide by gel filtration using Bio-Gel P-30 spin columns (Bio-Rad, Hercules, Calif.).

The following biotinylated gastrin peptide was used to form a tetramer: pEGPWLEEEEEAYGWMDFK($)# (SEQ ID NO:6), wherein #=amide; pE=pyroGlutamine and K($)=biotinylated Lysine.

Each tetramer was titrated in order to determine the optimal concentration.

Flow Cytometry

Cell suspensions of splenocytes were obtained by mechanical disruption of the spleens, and erythrocytes were lysed in 0.16 M chloride ammonium solution pH 7.4. The resulting single-cell suspensions were stained with combinations of fluorochrome-labeled antibodies and GnRH tetramers at optimal dilution. Staining was conducted at 4° C. in two steps. First, cells were stained with the PE-labeled tetramer for one hour, using the empirically determined optimal concentration. Cells were then washed three times with FACS buffer. Next, cells were stained with the APC- or FITC-labeled tetramer and combinations of PerCP-labeled anti-CD3 antibody (clone 145-2C11, BD Biosciences), FITC-labeled anti-CD19 antibody (clone 1D3, BD Biosciences), PE-labeled anti-CD27 antibody (clone LG.3A10, BD Biosciences) and PE-labeled CD138 antibody (clone 281-2, BD Biosciences). For the staining experiments involving directly labeled ligands, we used FITC- and biotin-labeled GnRH (Pepscan Systems).

Data were acquired using a FACSCalibur flow cytometer and CellQuest software (BD Immunocytometry Systems). One×10⁶ events were acquired per sample. For FACS analysis, lymphocytes were positively gated based on their forward and side scatter profile (live lymphocyte gate), and irrelevant T cells and auto-fluorescent cells were excluded with a negative gating on CD3-PerCP.

ELIspot assay. Nitrocellulose bottomed 96-well Multiscreen HA filtration plates (Millipore) were coated with 20 μg/ml of antigen or goat anti-mouse IgG antibody (SBA, Cat. No 1031-01) and incubated at 4° C. overnight. Wells were washed three times with PBS and blocked for 30 minutes with PBS 5% FCS. Blocking medium was replaced with 100 μl of RMPI medium 10% FCS and the resuspended cells from the cell cultures. Plates were incubated for 24 hours at 37° C. in incubator 6% CO₂. Wells were washed three times with PBS 0.05% Tween-20. 100 μl/well of AP-conjugated goat anti-mouse IgG (SBA, Cat. No. 1030-04) was added and incubated for two to four hours at room temperature. Wells were washed three times with PBA 0.05% Tween-20. 100 μl/well of BCIP/NBT substrate were added. After spot development, the plates were washed in tap water. The number of spots was evaluated using A.EL.VIS software.

Elisa test. 96-well multi-well plates were coated with 20μg/ml of antigen or goat anti-mouse IgG antibody (SBA, Cat. No. 1031-01) and incubated at 4° C. overnight. Wells were washed three times with PBS 0.05% Tween-20 and blocked for 30 minutes with PBS 5% BSA (Sigma). Wells were washed three times with PBS 0.05% Tween-20. In each well, the samples were added and incubated for one hour. Wells were washed three times with PBS 0.05% Tween-20. 100 μl/well of PO-conjugated goat anti-mouse IgG was added and incubated for one hour at room temperature. Wells were washed three times with PBA 0.05% Tween-20. 100 μl/well of ASBT substrate were added. After three minutes of incubation, the plates were read with an Elisa reader (Bio Rad).

Anti-peptide serum antibody level: radio-autoimmune assay. Antibody titers against GnRH were determined with a radio-immune assay (RIA) as described by Meloen et al. (1994). An amount of 50 μl antiserum diluted 1:1000 in PBS with 0.4% bovine serum albumin (BSA) was allowed to bind with iodinated GnRH (Amersham Pharmacia Biotech, Buckinghamshire, England) in 50 μl PBS with 0.4% BSA. After incubation for two days at 4° C., the iodinated GnRH bound to the antibodies was separated from the unbound using dextran-coated charcoal. The supernatant was counted and the percentage of iodinated GnRH bound by the antibodies in the 1:2000 diluted serum was calculated.

Results

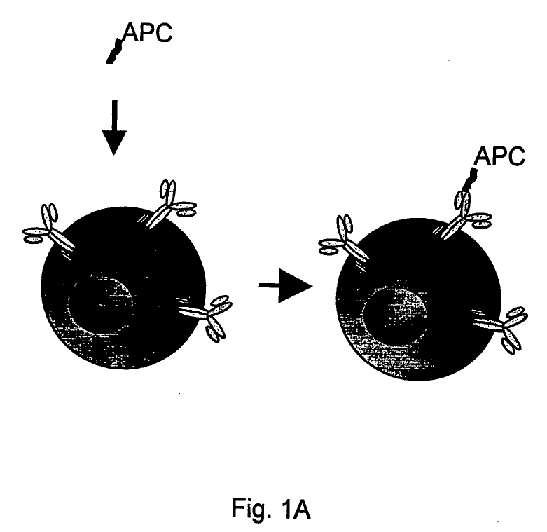

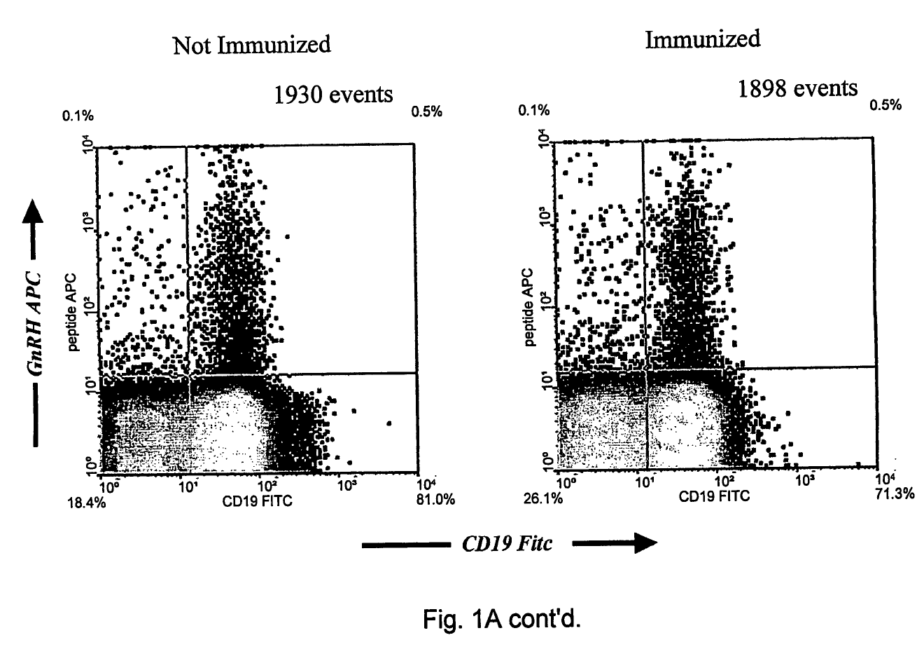

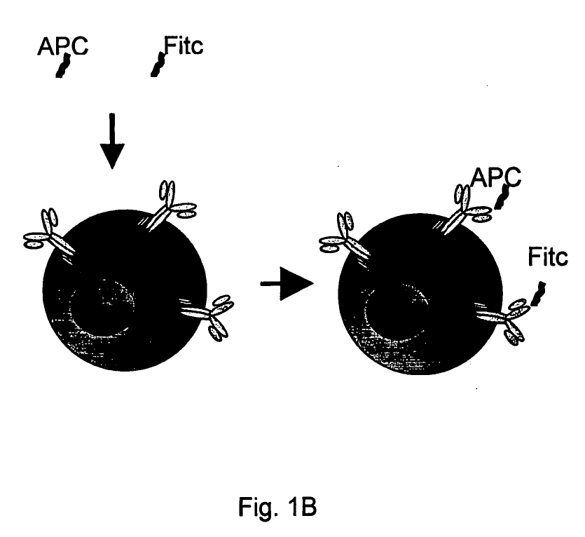

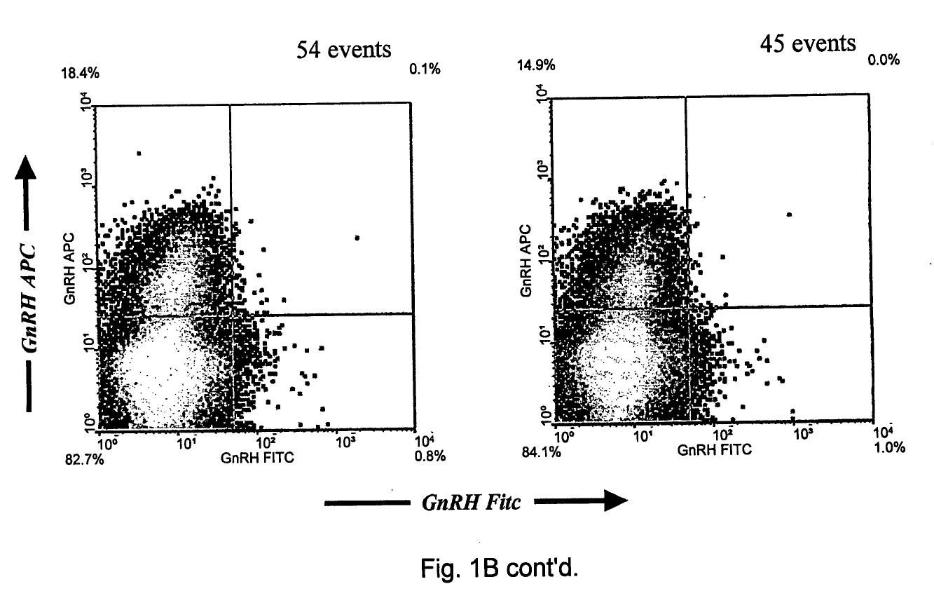

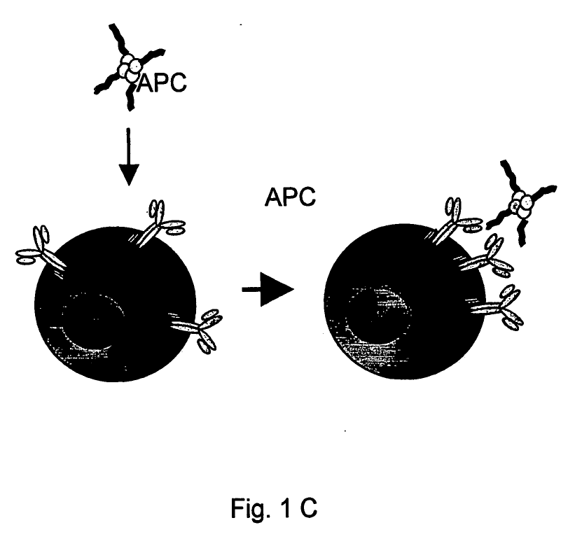

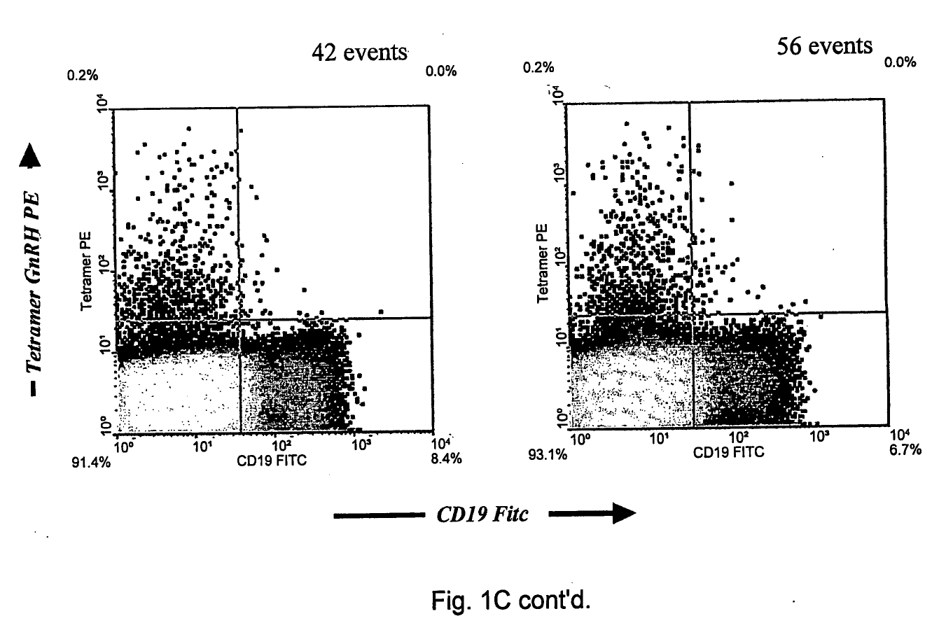

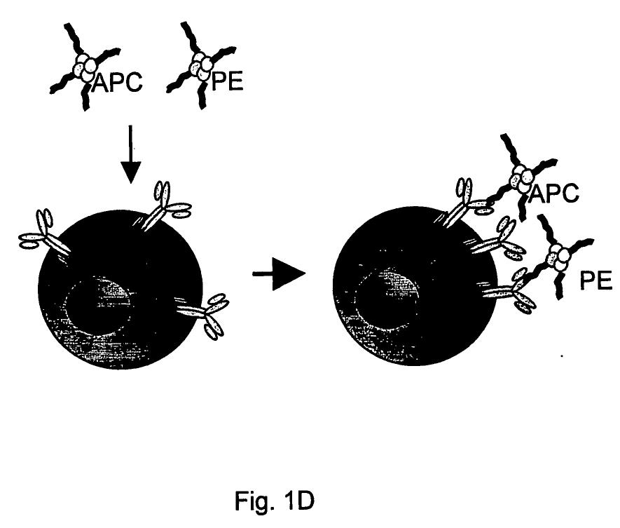

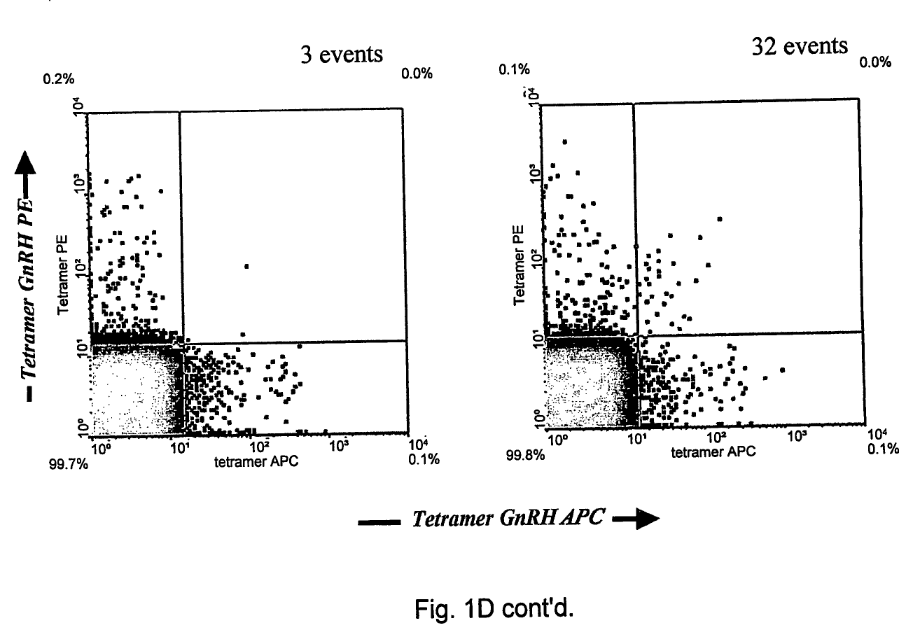

We developed a novel B-cell detection strategy able to lower the discrimination's level to 20 to 30 specific cells in one million of lymphocytes using tetrameric molecules. This method employed two different fluorescent-labeled molecules for each peptide, allowing improved signal-to-noise ratios and consequently, a very high sensitivity. To demonstrate the utility of our approach to detect ultra low-frequency B cells, we used the following as two model vaccine formulations: GnRH-like peptide and Gastrin-like peptide. Several methods have been described to detect antigen-specific B cells using flow cytometry (McHeyzer-Williams et al., 1993; Townsend et al., 2001; Newman et al., 2003), as shown schematically in FIG. 1.

To detect GnRH-specific B cells, we used directly labeled native GnRH or labeled tetramers with native GnRH, both in single- and double-staining protocols. GnRH tetramers were produced by incubating biotinylated GnRH with fluorescent-labeled streptavidin or neutravidin. Monoclonal antibodies against the B-cell marker CD19 and the T-cell marker CD3 were included in the staining procedure. The anti-CD3 staining was used to negatively select (irrelevant) T cells, as well as auto-fluorescent cells. As shown in FIG. 1, our results demonstrated that only double-tetramer staining produced discriminating results (FIG. 1D), and none of the other staining methods (FIGS. 1A-1C). Direct staining with labeled GnRH (FIGS. 1A, 1B) or with a single tetramer according to the method of Newman et al. (FIG. 1C) produced only high background signals and very little specific signal in the case of double-staining with labeled GnRH according to Townsend et al. (FIG. 1B). We found that the double-tetramer staining as disclosed in this application detects specific B cells at a frequency of 20 to 30 per 10⁶ splenocytes at seven days after a single immunization (FIG. 1D).

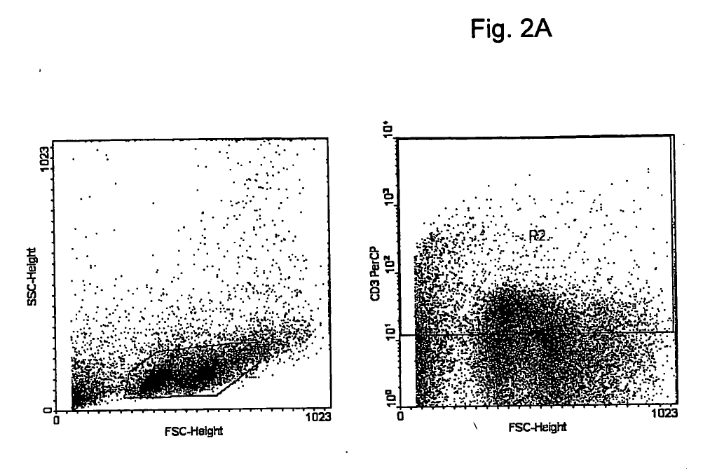

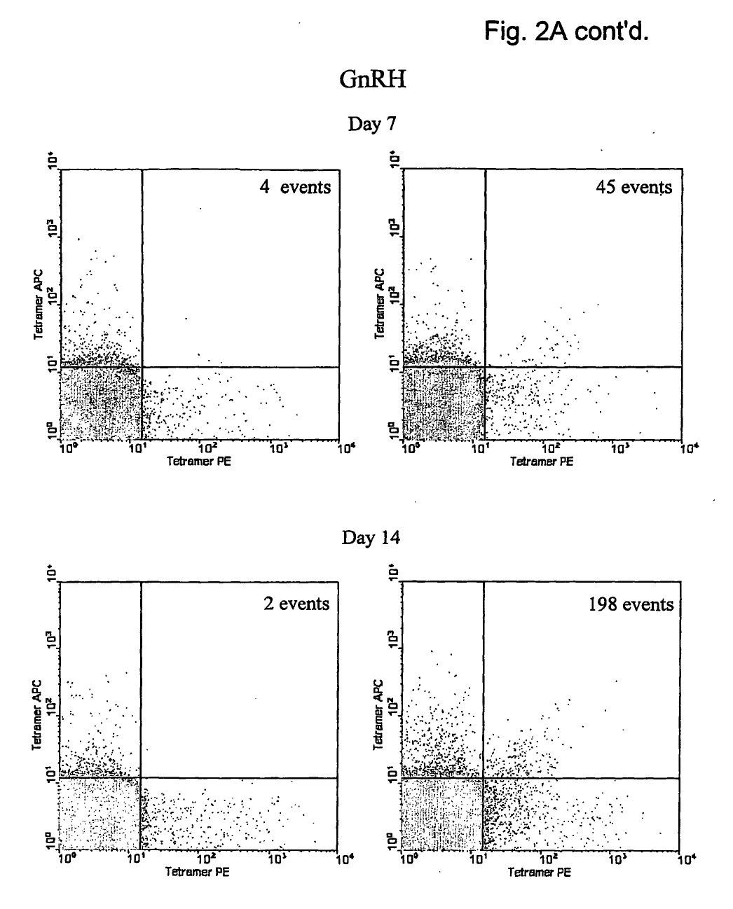

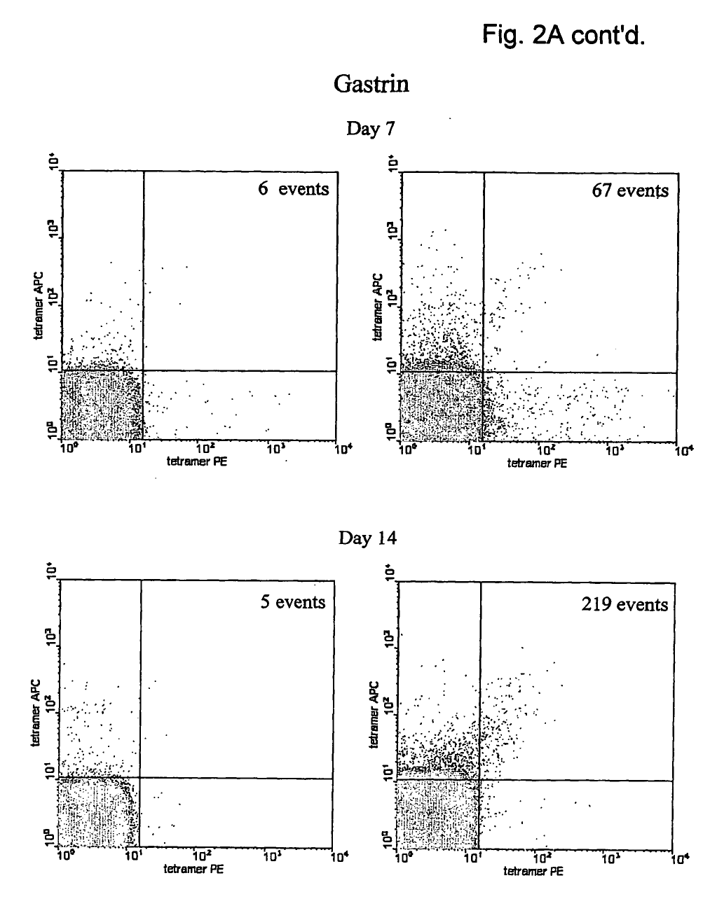

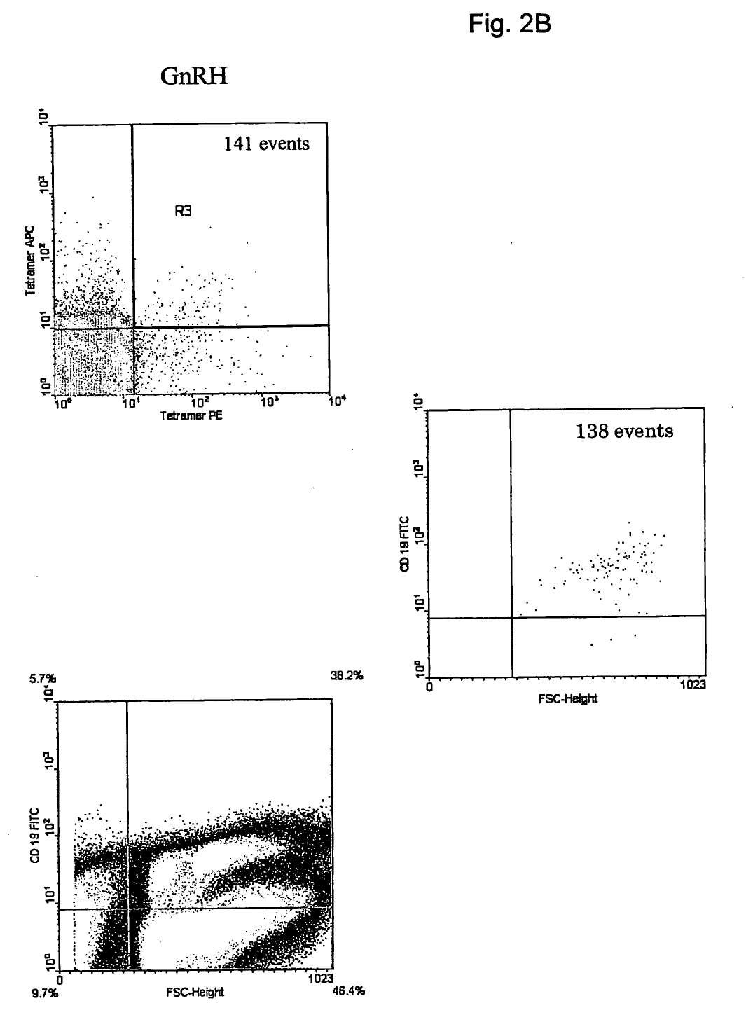

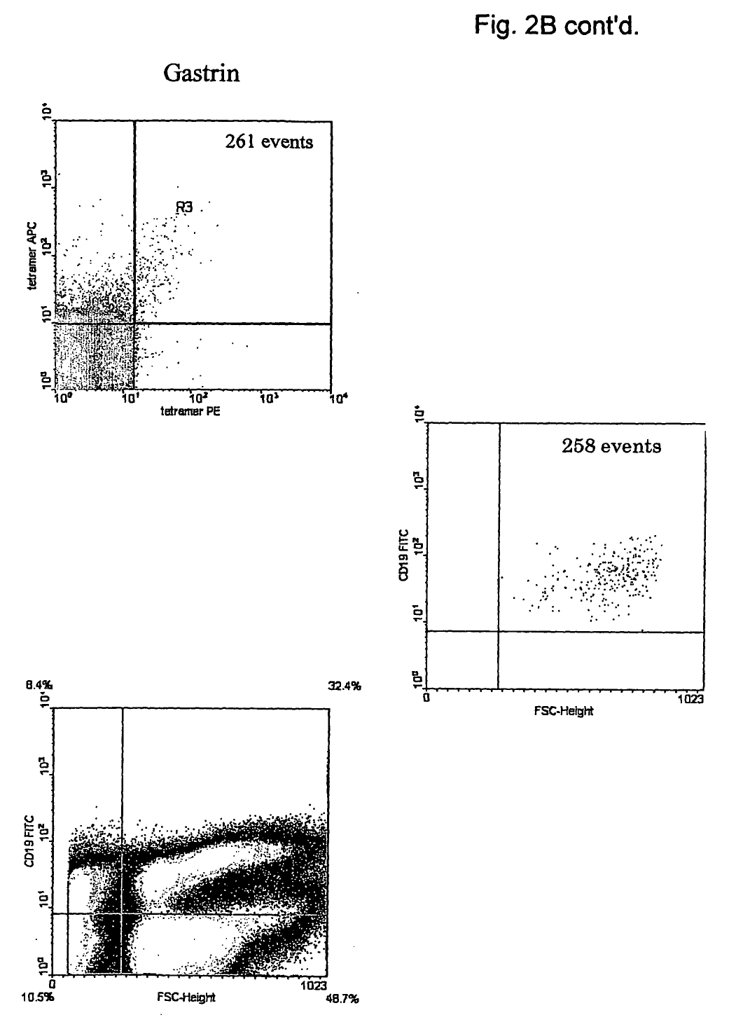

Lymphocytes were stained with peptide-tetramers and monoclonal antibody against CD19 and CD3. Since we expected only a very small population of specific B cells, debris were excluded with a live gate (forward and side scatter), and T cells were excluded with a negative gating on CD3e. Biotinylated GnRH tetrameric molecules or biotinylated gastrin tetrameric molecules were used in a double-staining strategy to identify peptide-specific cells (FIG. 2A). Using this approach, we were able to detect 20 to 30 specific peptide-tetramer double-positive cells in one million lymphocytes at day 7 after immunization, and 100 specific cells at day 14 after immunization (FIG. 2A).

After the discrimination of the peptide-tetramer double-positive cells, we studied the phenotypic characteristic of these cells. Although several authors have used different markers for the characterization of mature B cells, we decided to use CD19 as a pan B-cell marker (McHeyzer-Williams et al., 1993). All the double-positive cells were CD19-positive, classifying these cells as B cells. Interestingly, the rare double-positive cells found in the non-immunized mice are also CD19-positive (FIG. 2B), showing that these cells are the B-cell precursor population for the specific antigen that comprises approximately three to four cells per million of lymphocytes in naive mice.

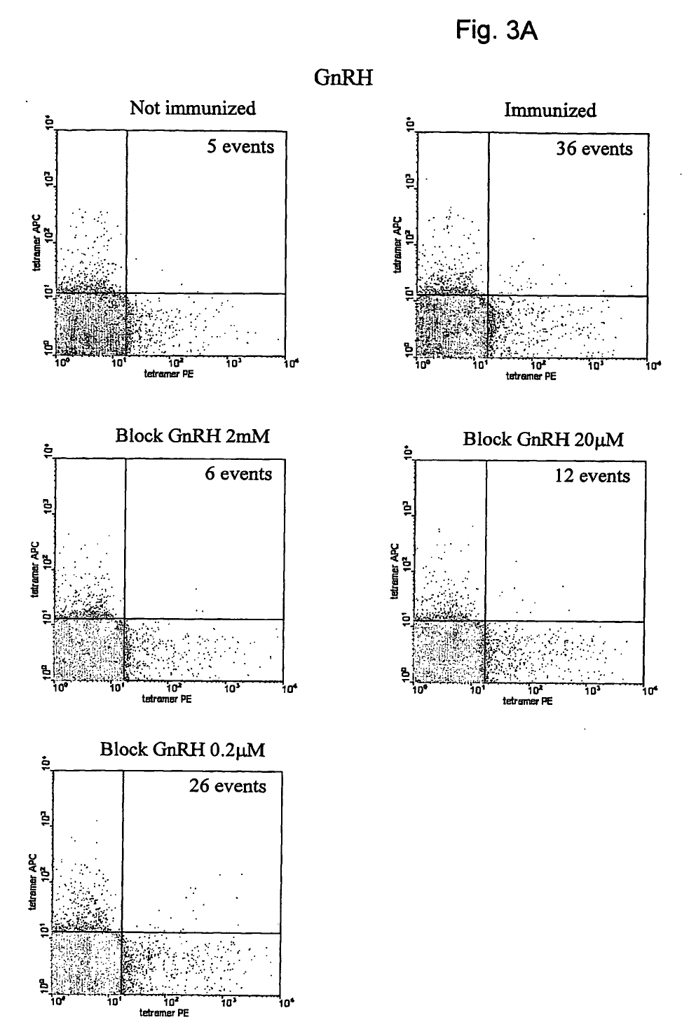

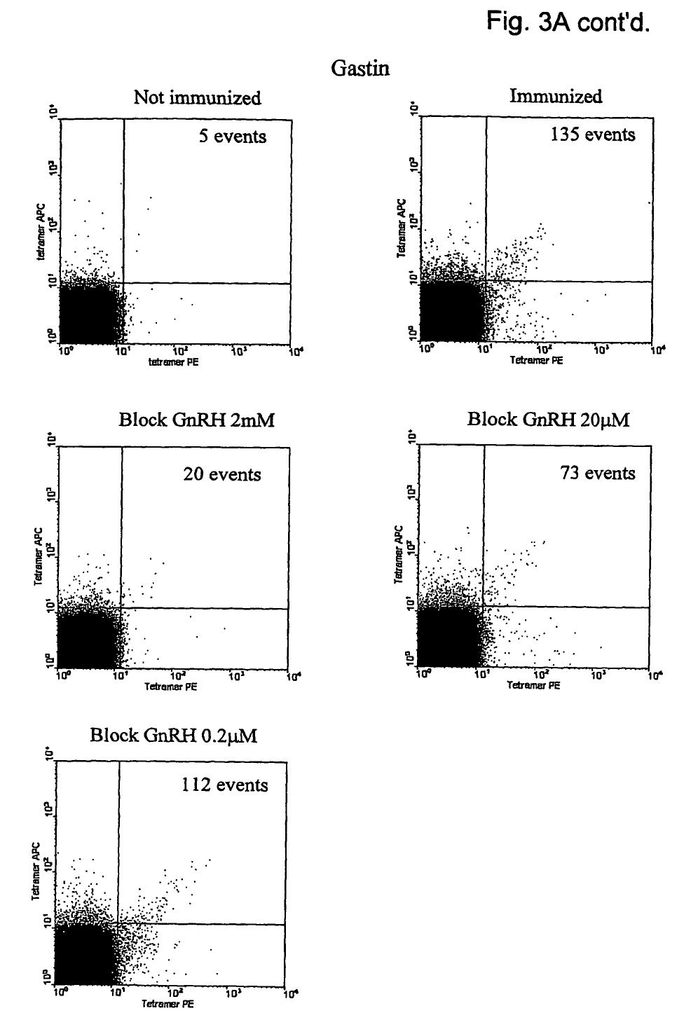

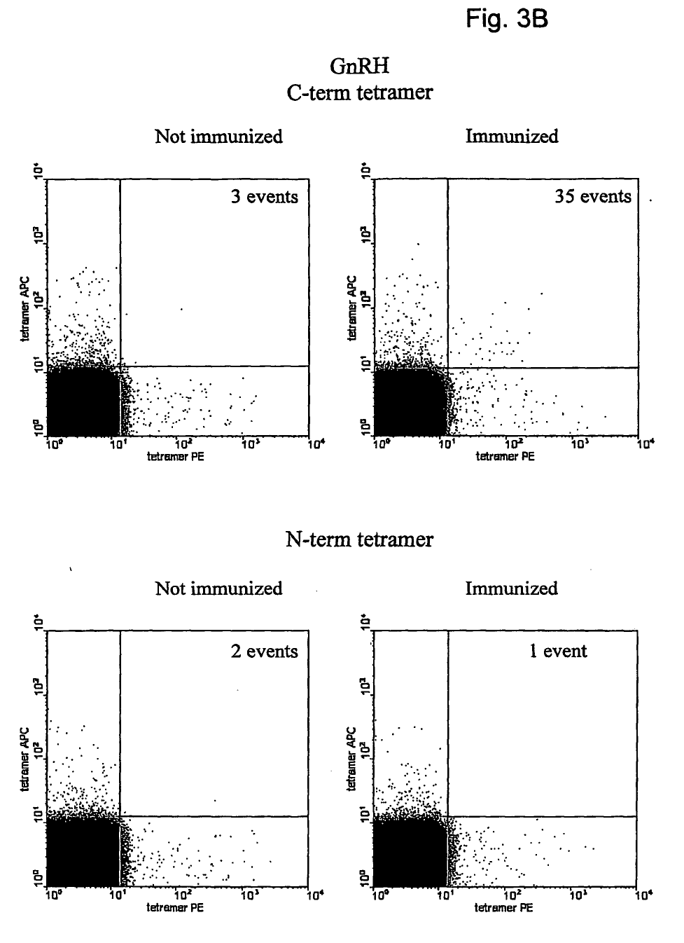

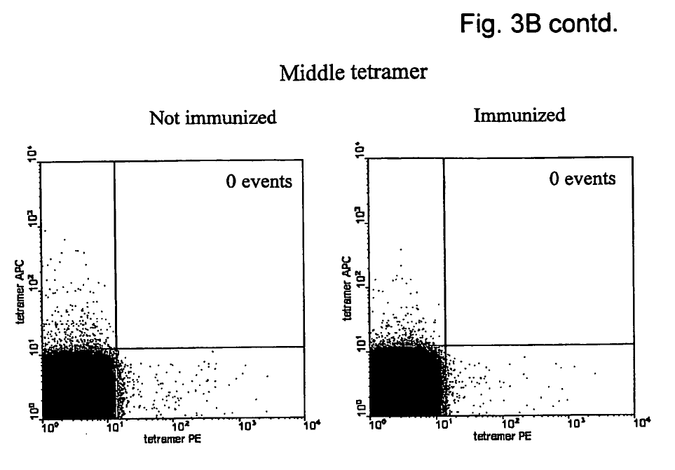

The specificity of tetramer binding was demonstrated by inhibition studies with unlabeled GnRH (see FIG. 3A) or Gastrin (data not shown). Lymphocytes were preincubated with unlabeled peptide followed by incubation with labeled tetramers, CD19 and CD3e antibodies. As shown in FIG. 3A, tetramer staining was inhibited by inclusion of unlabeled peptide in a dose-dependent manner.

As a further specificity test, we produced tetramers using peptides that were N-, middle-, or C-terminally labeled. As shown in FIG. 3B, we found that only the C-term biotinylated GnRH tetrameric was able to detect the specific B cells.

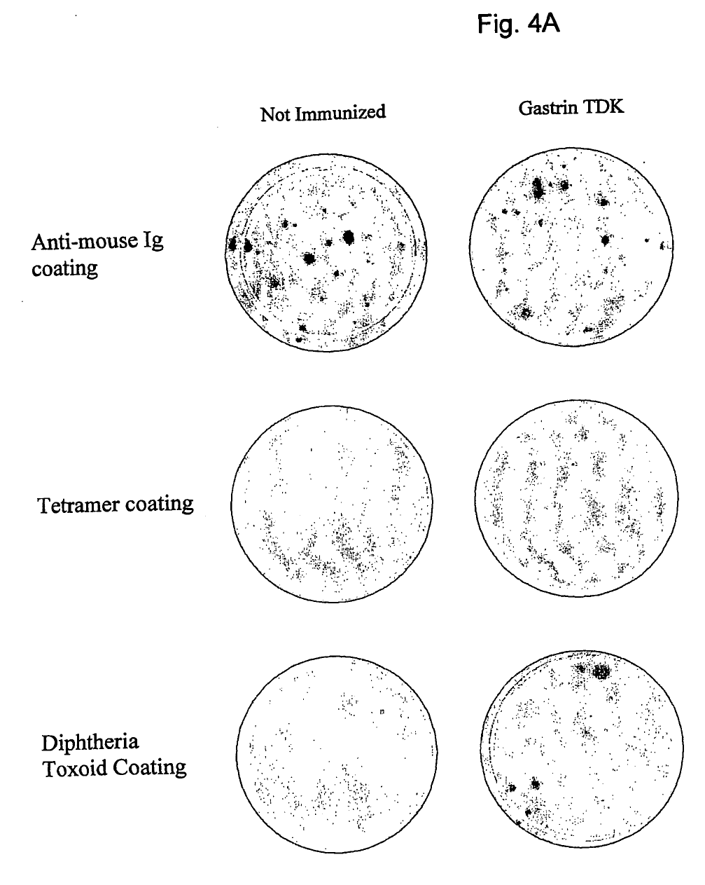

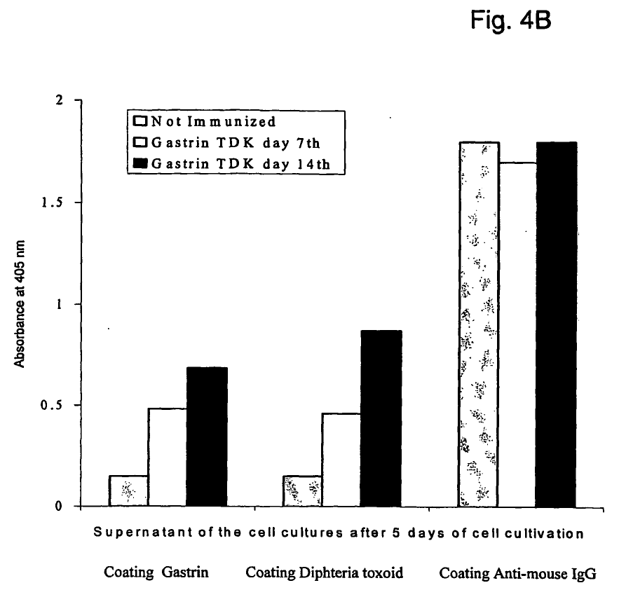

After removing the erythrocytes from the spleen samples, splenocytes from gastrin-immunized mice were cultivated for five days under IL-2 and Pansorbin stimulation. At the end of this time, the supernatants were used for performing an ELISA test, and the cells were used in an ELIspot assay. As shown in FIG. 4A, only the cells of the immunized mice were able to produce spots on the ELlspot well. These results were confirmed by ELISA test (FIG. 4B) and they are consistent with the results of FACS analysis, proving that B-cell detection is a reliable method for selecting peptide vaccines.

Therefore, in the present study, we have disclosed a novel method to visualize antigen-specific B cells at very low frequencies. In our experiments, the double-tetramer staining procedure was more sensitive and more reproducible than the previously described methods (e.g., labeled peptide ligands, single tetramers). Our peptide tetramers have proven to be highly specific B-cell staining reagents.

### EXAMPLE 2

Comparison of the Immunogenicity of GnRH-Derived Peptides.

Materials and Methods

Animals

Six eight-week-old mice were used and treated according to the ethical guidelines of the University of Utrecht. Mice were immunized intraperitoneally on day 0 with GnRH-like peptides conjugated to ovalbumin (OVA). We prepared three different peptides, i.e., GnRH-monomer (pEHWSYGLRPGC (SEQ ID NO:1)), GnRH-tandem (pEHWSYGLRPGQHWSYGLRPGC (SEQ ID NO:2)) and GnRH-tandem dimer (TDK, the dimerized form of pEHWSYkLRPGQHWSYkLRPGC in which “k” represents a D-lysine residue) (Pepscan Systems, Lelystad, NL), in Complete Freunds Adjuvant. Control mice were not immunized. On day 7, half of the mice of each group were boosted, using Incomplete Freunds Adjuvant, and half of the animals were sacrificed and spleens were harvested. On day 14, spleens were harvested from the remaining mice. All animal experiments were done with permission from the local ethical committee (DEC).

Cell cultivation. Cell suspensions of splenocytes were cleared of erythrocytes and the cells were cultured in T75 flasks in the presence of Pansorbin (SAC, Calbiochem, Cat. No. 507858) and recombinant human IL-2 (CETUS) for five days in incubator at 37° C. and 5% CO₂. Cells were cultivated 5 to 10×10⁷ spleen cells at 2×10⁶/ml in RMPI 10% FCS. After the incubation, the cells were collected in tubes and centrifuged at 1200 rpm for ten minutes. Supernatants were used for Elisa tests.

Tetramer synthesis. Tetrameric molecules were produced by mixing an equal amount of C-term biotinylated peptide at 200 μM (Pepscan B. V.) with R-phycoerythrin (PE)-labeled neutravidin at 3.3 μM (PE-NA, Molecular Probes, Eugene, Oreg.), or allophycocyanin (APC)-labeled streptavidin at 6.1 μM (APC-SA, Molecular Probes). Each mixture was incubated at 4° C. for a minimum of 12 hours. Then, tetramers were separated from non-complexed peptide by gel filtration using a Bio-Gel P-30 spin column (Bio-Rad, Hercules, Calif.). The amount of tetramers to use was determined by titration experiments. Peptides used were derived from GnRH (Pepscan B.V.).

Flow cytometry. Cell suspension of splenocytes was obtained by mechanical disruption of the spleens, and erythrocytes were lysed in 0.16 M chloride ammonium solution pH 7.4. The resultant cell suspensions were stained with combinations of fluorochrome-labeled antibodies and peptide's tetrameric molecules at optimal dilution. Staining was routinely conducted at 4° C. in two steps: first, with peptide's tetrameric molecule labeled with PE for one hour. Cells were then washed three times with FACS Buffer. Next, cells were stained with the APC- or FITC-labeled tetramer and combinations of PerCP-labeled anti-CD3 antibody (clone 145-2C11, BD Biosciences) and FITC-labeled anti-CD19 antibody (clone 1D3, BD Biosciences) were used for the second step. Data were acquired using a FACSCalibur flow cytometer and Cell Quest software (BD Immunocytometry Systems). One×10⁶ events were acquired per sample. All data are representative plots derived from a minimum of three independent experiments in which at least three experimental and three control mice were analyzed.

Results

To detect GnRH-specific B cells, we used labeled tetramers with native GnRH, in double-staining protocols. Monoclonal antibodies against the B-cell marker CD19 and the T-cell marker CD3 were included in the staining procedure. The anti-CD3 staining was used to negatively select (irrelevant) T cells, as well as auto-fluorescent cells.

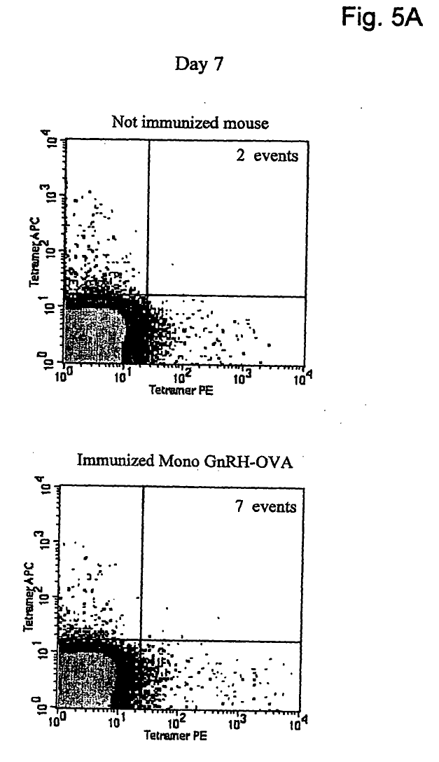

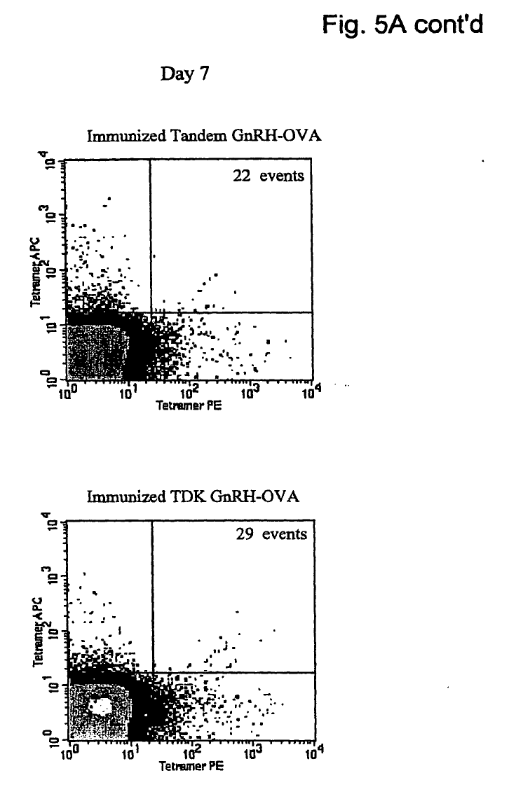

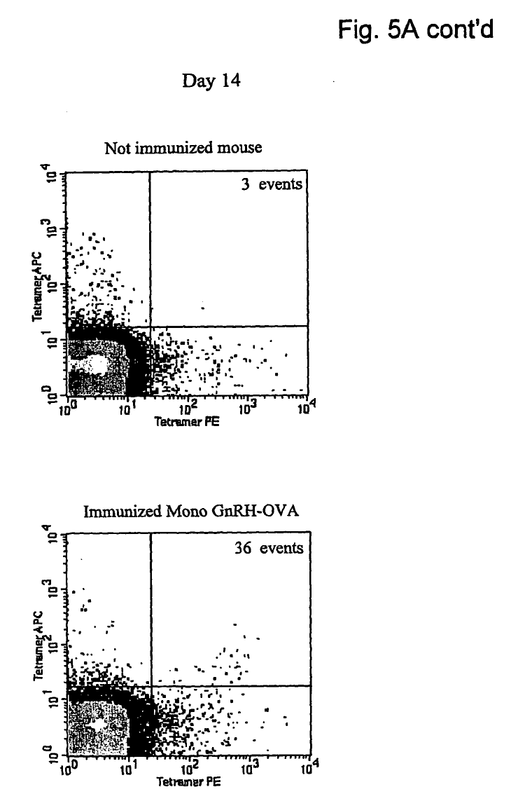

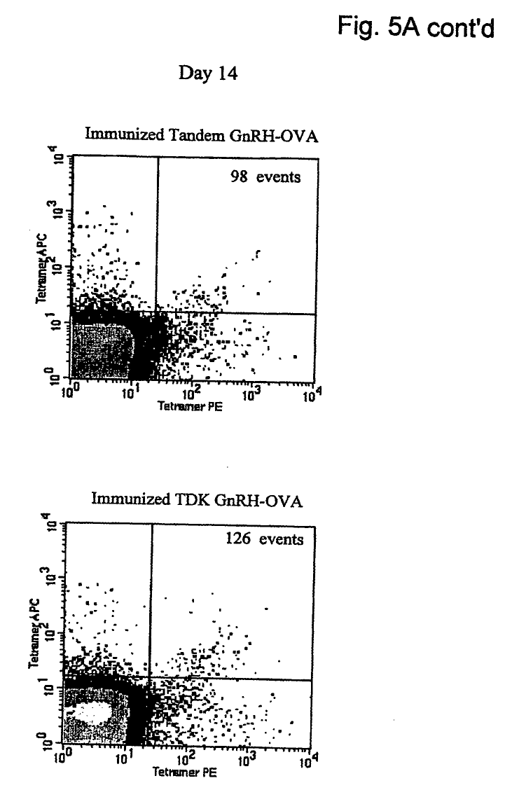

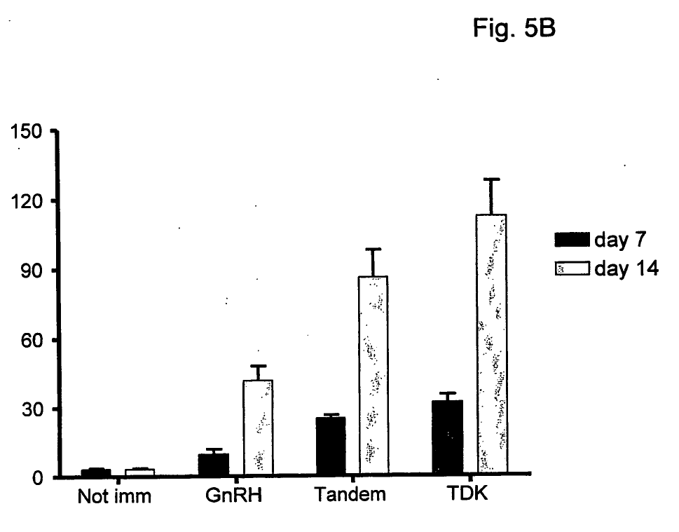

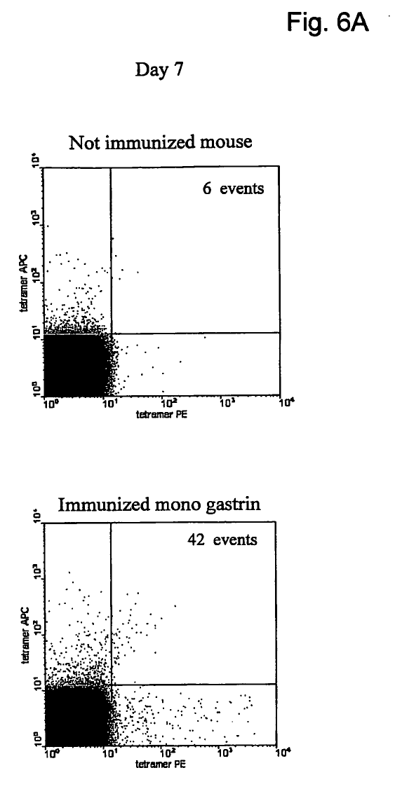

With the above-described method, we compared the three different peptide formulations of GnRH to vaccinate the mice. The results of the double-tetramer staining assay are given in FIGS. 5A and 5B. The results clearly show that GnRH TDK-OVA caused the most extensive B-cell expansion, which was measured at both seven and 14 days after vaccination.

### EXAMPLE 3

Comparison of the Immunogenicity of Gastrin-Derived Peptides.

With the above-described method, we compared four different peptide formulations of Gastrin to vaccinate the mice.

| | |
|---|---|
| mono Gastrin: | |
| pEGPWLEEEEC#; | (SEQ ID NO:7) |
| tandem Gastrin: | |
| pEGPWLEEEEQGPWLEEEEC#; | (SEQ ID NO:8) |
| Gastrin TDK 1: | |
| pEGPWLEEEEQGPWLEEEECK# | (see, SEQ ID NO:8) |
| \| | |
| pEGPWLEEEEQGPWLEEEECK# | |
| and | |
| Gastrin TDK 2: | |
| pEGPWLEEEEEAYkQGPWLEEEEEAYkC# | (see, SEQ ID NO:8) |
| \| | |
| pEGPWLEEEEEAYkQGPWLEEEEEAYkC# | |

These peptides were tested as a vaccine in mice and at seven and 14 days after immunization, specific B cells were detected with the method of the invention using tetramer Gastrin peptide. The following biotinylated gastrin peptide was used to form a tetramer: pEGPWLEEEEEAYGWMDFK($)# (SEQ ID NO:6), wherein #=amide; pE=pyroGlutamine and K($)=biotinylated Lysine.

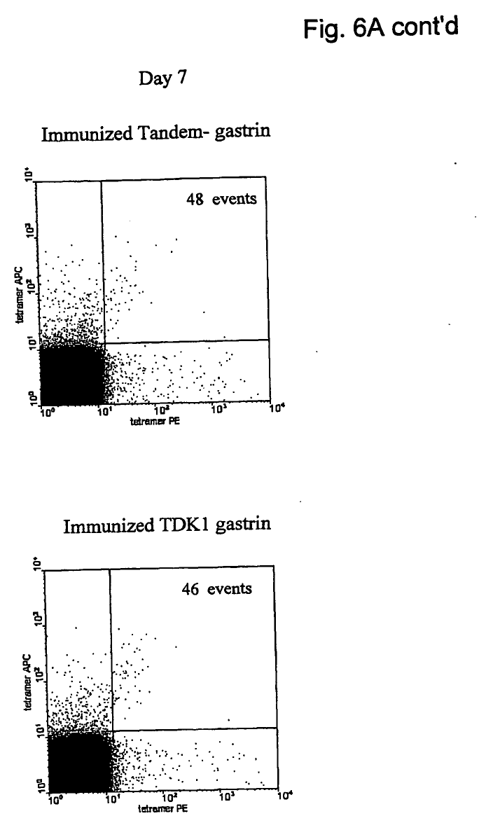

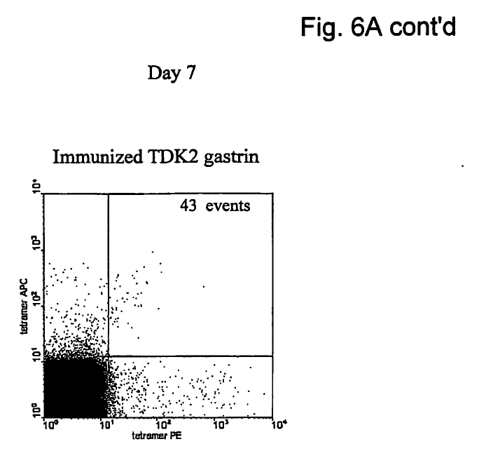

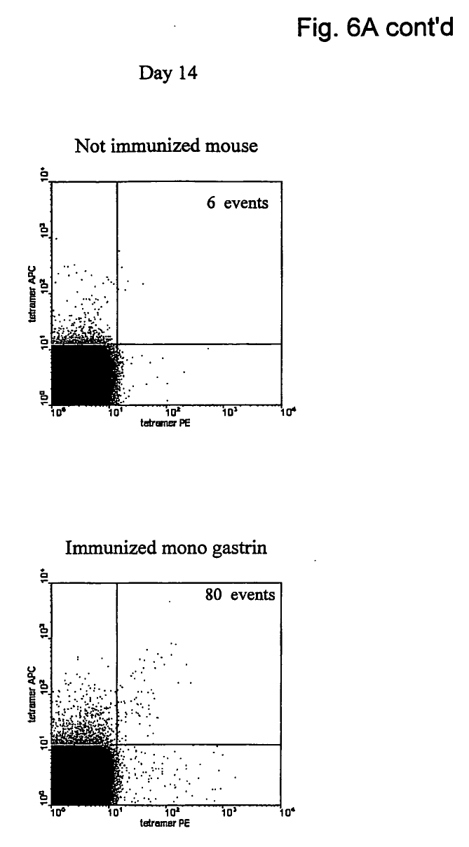

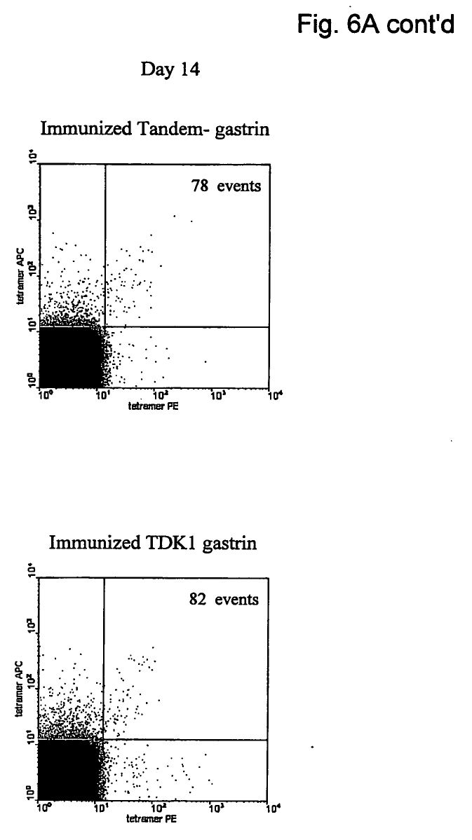

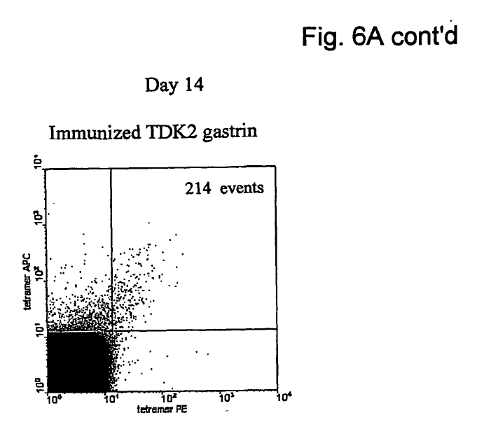

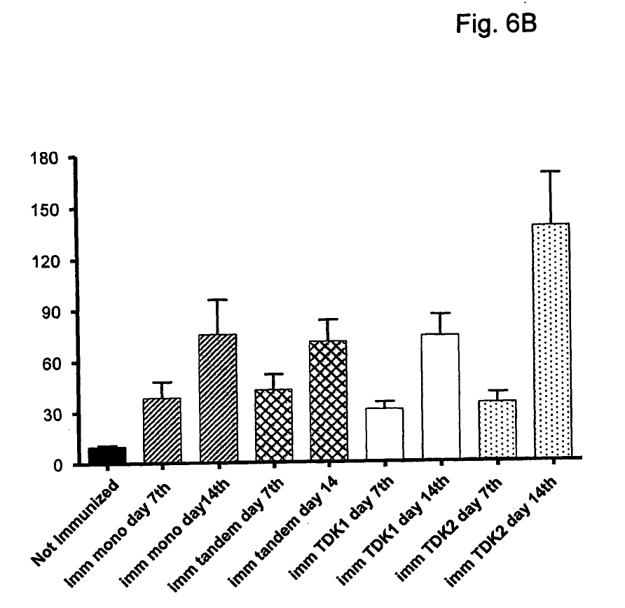

The results of the double-tetramer staining assay are presented in FIGS. 6A and 6B. The results clearly show that Gastrin TDK2-OVA caused the most extensive B-cell expansion, which was measured at 14 days after vaccination.

### EXAMPLE 4

Detection of Low-Frequency Early B Cells in Rats Immunized with a C-Terminal Peptide of hCG.

Materials and Methods

Animals

Six eight-week-old rats were used and were treated according to the ethical guidelines of our institutions. Rats (nos. 1, 2, and 3) were immunized intraperitoneally on day 0 with 100 μg C-Terminal Peptide (CTP), which comprises amino acid position 109-145 of hCG, coupled with 300 μg KLH, in Complete Freunds Adjuvant (CFA). The accession number of hCG is p01233, the Swiss prot. entry name is CGHB_HUMAN.

Control rats (nos. 4, 5, and 6) were immunized with KLH in CFA. On days 10 to 12, the animals were sacrificed and spleens were harvested. All animal experiments were performed with permission from the local ethical committee (DEC).

Flow Cytometry

Cell suspensions of splenocytes were obtained by mechanical disruption of the spleens. Lymphocytes were collected using a Ficoll gradient. The resulting single-cell suspensions were stained with hCG-tetramers at optimal dilution. Staining was conducted at 4° C. in two steps. First, cells were stained with the PE-labeled tetramer of hCG for one hour, using the empirically determined optimal concentration. Cells were then washed three times with FACS buffer. Next, cells were stained with the APC-labeled tetramer of hCG and washed again.

Data were acquired using a FACSCalibur flow cytometer and CellQuest software (BD Immunocytometry Systems). One×10⁶ events were acquired per sample. For FACS analysis, lymphocytes were positively gated based on their forward and side scatter profile (live lymphocyte gate).

Results

The method of the invention detected, within 12 days in an animal that was immunized only once, a total of 136 specific cells in 1×10⁶ splenocytes using tetrameric molecules. This method employed two different fluorescent-labeled molecules for hCG, allowing improved signal-to-noise ratios and consequently, a very high sensitivity.

To detect hCG-specific B cells, we used labeled tetramers with native hCG, in a double-staining protocol.

TABLE 1
Results of flow cytometry of rat lymphocytes after immunization with
CTP-hCG.

| | | Pos. B cells per |
|---|---|---|
| Rat no. | Inoculation | 1 × 10⁶ cells in Facs |
| 1 | CTP-KLH/CFA | 61 |
| 2 | CTP-KLH/CFA | 47 |
| 3 | CTP-KLH/CFA | 136 |
| 4 | KLH/CFA | 52 |
| 5 | KLH/CFA | 58 |
| 6 | KLH/CFA | 100 |

Lymphocytes were stained with hCG-tetramers. Since we expected only a very small population of specific B cells, debris was excluded with a live gate (forward and side scatter). hCG-trameric molecules were used in a double-staining strategy to identify hCG-specific cells (Table 1). Using this approach, we were able to detect 136 specific hCG-tetramer double-positive cells in one million splenocytes at day 12 after immunization. Only the three rats was detected as a responder to the immunization based on specific B-cell responses.

Therefore, in this example, we have disclosed that the novel method to visualize antigen-specific cells at very low frequencies also functions for protein tetramers. Our protein tetramers have proven to be highly specific B-cell staining reagents.

### REFERENCES

Adams, C. L., M. K. L. MacLeod, E. J. Milner-White, R. Aitken, P. Garside and D. I. Stott. Complete analysis of the B-cell response to a protein antigen, from in vivo germinal center formation to 3D-modeling affinity maturation. Immunology 108, 274-287 (2003).
Altman, J. D., P. A. H. Moss, P. J. R. Goulder, D. H. Barouch, M. McHeyzer-Williams, J. Bell, A. J. McMichael and M. M. Davis. Phenotypic analysis of antigen-specific lymphocytes. Science 274, 94-96 (1996).
Bateman, H. E., B. S. Kasimis, C. R. Yook and V. Hiremath. 1995. Case report: primary choriocarcinoma of the stomach. N. J. Med. 92:459-462.
Carpelan-Holmstrom, M., C. Haglund, J. Lundin, H. Alfthan, U. H. Stenman and P. J. Roberts. 1996. Independent prognostic value of preoperative serum markers CA 242, specific tissue polypeptide antigen and human chorionic gonadotrophin beta, but not of carcinoembryonic antigen or tissue polypeptide antigen in colorectal cancer. Br. J. Cancer 74:925-929.
Cauchi, M. N., S. H. Koh, D. Lim and D. L. Hay. 1981. Oncofoetal antigens in cancer of the cervix and ovary. Br. J. Cancer 44:403-409.
Davis, M. M., and P. J. Bjorkman. T-cell antigen receptor genes and T-cell recognition. Nature 334, 395 (1988).
Fuerst, J., E. Fiebiger, A. Jungwirth, D. Mack, P. G. Talwar, J. Frick and E. Rovan. Effect of active immunization against LHRH on androgen-sensitive Dunning R3327-PAP and androgen-independent Dunning R3327-AT2.1 prostate cancer cell lines. Prostate 32, 77-84 (1997).
Fujimaki, T., K. Mishima, A. Asai, K. Tabuchi, M. Kobayashi, I. Suzuki and T. Kirino. 2000. Levels of beta-human chorionic gonadotropin in cerebrospinal fluid of patients with malignant germ cell tumor can be used to detect early recurrence and monitor the response to treatment. Jpn. J. Clin. Oncol. 30:291-294.
Hara, I., Y. Yamada, H. Miyake, S. Hara, A. Gotoh, M. Fujisawa, H. Okada, S. Arakawa and S. Kamidono. 2002. Detection of beta-human chorionic gonadotropin-expressing cells by nested reverse transcriptase-polymerase chain reaction in the peripheral blood stem cells of patients with advanced germ cell tumor. J. Urol. 167:1487-1491.
Higashida, T., T. Koizumi, S. Yamaguchi, T. Ichimura, K. Hasegawa and R. Nishimura. 2001. Ovarian malignant mixed mesodermal tumor producing the free form of the beta-subunit of human chorionic gonadotropin. Int. J. Clin. Oncol. 6:97-100.
Hoermann, R., A. L. Gerbes, G. Spoettl, D. Jungst and K. Mann. 1992. Immunoreactive human chorionic gonadotropin and its free beta subunit in serum and ascites of patients with malignant tumors. Cancer Res. 52:1520-1524.
Hoosein, N. M., P. A. Kiener, R. C. Curry and M. G. Brattain. Evidence for autocrine growth stimulation of cultured colon tumor cells by a Gastrin cholecystokinin-like peptide. Exp. Cel. Res. 186, 15-21 (1990).
Hotakainen, K., C. Haglund, A. Paju, S. Nordling, H. Alfthan, E. Rintala and U. H. Stenman. 2002. Chorionic Gonadotropin beta-Subunit and Core Fragment in Bladder Cancer: mRNA and Protein Expression in Urine, Serum and Tissue. Eur. Urol. 41:677-685.
Hotakainen, K., B. Ljungberg, A. Paju, T. Rasmuson, H. Alfthan and U. H. Stenman. 2002. The free beta-subunit of human chorionic gonadotropin as a prognostic factor in renal cell carcinoma. Br. J. Cancer 86:185-189.
Jackson, D. C., A. W. Purcell, C. J. Fitzmaurice, W. Zeng and D. N. J. Hart. The central role played by peptides in the immune response and the design of peptide-based vaccines against infectious diseases and cancer. Current Drug Targets 3, 175-196 (2002).
Jensen, R. T. Involvement of cholecystokinin Gastrin-related peptides and their receptors in clinical gastrointestinal disorders. Pharmacology and Toxicology 91, 333-350 (2002).
Koh, T. J., G. J. Dockray, A. Varro, R. J. Cahill, C. A. Dangler, J. G. Fox and T. C. Wang. Overexpression of glycine-extended Gastrin in transgenic mice results in increased colonic proliferation. J. Clin. Invest. 103, 1119-1126 (1999).
Kouskoff, V., G. Lacaud, K. Pape, M. Retter and D. Nemazee. B-cell receptor expression level determines the fate of developing B lymphocytes: receptor editing versus selection. PNAS. 13, 743507439 (2000).
Liu, E., N. Abiru, H. Moriyama, D. Miao and G. S. Eisenbarth. Induction of insulin auto-antibodies and protection from diabetes with subcutaneous insulin B:9-23 peptide without adjuvant. Ann. N. Y. Acad. Sci. 958, 224-7 (2002).
Lundin, M., S. Nordling, M. Carpelan-Holmstrom, J. Louhimo, H. Alfthan, U. H. Stenman and C. Haglund. 2000. A comparison of serum and tissue hCG beta as prognostic markers in colorectal cancer. Anticancer Res. 20:4949-4951.
Manabe, T., M. Adachi and K. Hirao. 1985. Human chorionic gonadotropin in normal, inflammatory, and carcinomatous gastric tissue. Gastroenterology 89:1319-1325.
McHeyzer-Williams, M. G., M. J. McLean, P. A. Lalor and G. J. V. Nossal. Antigen-driven B-cell differentiation in vivo. J. Exp. Med. 178(1), 295-307 (1993).
Meloen, R. H., J. P. M. Langeveld, W. M. M. Schaaper and J. W. Slootstra. Synthetic peptide vaccines: unexpected fulfillment of discarded hope? Biologicals 29, 233-236 (2001).
Monzavi-Karbassi, B., G. Cunto-Amesty, P. Luo and T. Kieber-Emmons. Peptide mimotopes as surrogate antigens of carbohydrates in vaccine discovery. Trends Biotechnol. 20(5), 207-14 (2002).
Meyer, T., L. A. Cole, P. I. Richman, H. D. Mitchell, J. Myers and G. J. Rustin. 2001. High levels of hCG in choriocarcinoma can result in renal failure and a false-negative pregnancy test in men. Clin. Oncol. (R. Coll. Radiol.) 13:301-303.
Newman, J., J. S. Rice, C. Wang, S. L. Harris and B. Diamond. Identification of an antigen-specific B-cell population. Journal of Immunological Methods 272, 177-187 (2003).
Nicoll, J. A., D. Wilkinson, C. Holmes, P. Steart, H. Markham and R. O. Weller. Neuropathology of human Alzheimer disease after immunization with amyloid-beta peptide: a case report. Nat. Med. 9(4), 448-52 (2003).
Noguchi, M., K. Kobayashi, N. Suetsugu, K. Tomiyasu, S. Suekane, A. Yamada, K. Itoh and S. Noda. Induction of cellular and humoral immune responses to tumor cells and peptides in HLA-A24 positive hormone-refractory prostate cancer patients by peptide vaccination. Prostate 57(1), 80-92 (2003).
Oonk, H. B., J. A. Turkstra, W. M. M. Schaaper, J. H. F. Erkens, M. H. Schuitemaker de Weerd, A. Van Nes, J. H. M. Verheijden and R. H. Meloen. New GnRH-like peptide construct to optimize efficient immunocastration of male pigs by immunoneutralization of GnRH. Vaccine 16, 1074-1082 (1998).
Ribas, A., J. M. Timmerman, L. H. Butterfield and J. S. Economou. Determinant spreading and tumor responses after peptide-based cancer immunotherapy. Trends Immunol. 24(2), 58-61 (2003).
Smith, A. M., T. Justin, D. Michaeli and S. Watson. Phase II/I study of G17-DT, an anti-gastrin immunogen, in advanced colorectal cancer. Clin. Canc. Res. 6, 4719-4724 (2000).
Stetson, D. B., M. Mohrs, V. Mallet-Designe, L. Teyton and R. M. Locksley. Rapid expansion and IL-4 expression by Leishmania-specific naïve helper T cells in vivo. Immunity 17, 191-200 (2002).
Syrigos, K. N., I. Fyssas, M. M. Konstandoulakis, K. J. Harrington, S. Papadopoulos, N. Milingos, P. Peveretos and B. C. Golematis. 1998. Beta human chorionic gonadotropin concentrations in serum of patients with pancreatic adenocarcinoma. Gut. 42:88-91.
Szavay, P. O., C. Wermes, J. Fuchs, M. Schrappe, P. Flemming and D. von Schweinitz. 2000. Effective treatment of infantile choriocarcinoma in the liver with chemotherapy and surgical resection: a case report. J. Pediatr. Surg. 35:1134-1135.
Talwar, G. P. Vaccines and passive immunological approaches for the control of fertility and hormone-dependent cancers. Immunological review 171, 173-192 (1999).
Thorburn, C. M., G. D. Friedman, C. J. Dickinson, J. H. Vogelman, N. Orentreich and J. Parsonnet. Gastrin and colorectal cancer: a prospective study. Gastroenterology 115, 275-280 (1998).
Townsend, S. E., C. C. Goodnow and R. J. Cornall. Single epitope multiple staining to detect ultra low-frequency B cells. Journal of Immunological Methods 249, 137-146 (2001).
Uckaya, G., G. Uckaya, A. Ozet, A. Arpaci and S. Komurcu. 1998. Human chorionic gonadotropin and CA 15-3 producing adenocarcinoma. Nuklearmedizin 37:297-298.
Watson, S., L. Durrant and D. Morris. Gastrin: growth-enhancing effects on human gastric and colonic tumor cells. Br. J. Cancer 59, 554-558 (1989).

1. A method for detecting a cell comprising a member of an affinity binding pair, said member being specific for an epitope in a proteinaceous molecule, the method comprising:
generating a synthetic peptide derived from at least part of said proteinaceous molecule,
immunizing an animal with said synthetic peptide, and
detecting in a sample obtained from the animal, a cell comprising a member of an affinity binding pair that is specific for said proteinaceous molecule.

2. The method according to claim 1, wherein said sample is incubated with a first molecule comprising at least two copies of said epitope from said proteinaceous molecule or an equivalent thereof, comprising a first label, and a second molecule comprising at least two copies of said epitope from said proteinaceous molecule or an equivalent thereof, comprising a second label and detecting in said sample a cell that comprises said first and said second labels.

3. The method according to claim 1, wherein said synthetic peptide is a modified peptide that is immunologically different than any peptide of said proteinaceous molecule.

4. The method according to claim 1, wherein said synthetic peptide comprises a dimer peptide, a tandem dimer peptide, a mimotope, a replacement-net mapped peptide, and/or an amino acid substituted peptide, comprising between 1 and 6 amino acid substitutions per 20 amino acids of said peptide.

5. The method according to claim 1, wherein the proteinaceous molecule is selected from the group consisting of GnRH, gastrin, hCG, and PTHrP.

6. The method according to claim 1, wherein said cell is an immune cell.

7. The method according to claim 5, wherein said immune cell is a B-cell.

8. The method according to claim 1, wherein said first and second molecules comprise tetramers of said epitope or tetramers of said proteinaceous molecule.

9. A composition comprising:
a first molecule comprising a tetramer or tetramers of an epitope or a tetramer or tetramers of a proteinaceous molecule, said first molecule associated with a first label, and
a second molecule comprising a tetramer or tetramers of an epitope or a tetramer or tetramers of a proteinaceous molecule, said second molecule associated with a second label,
wherein a signal obtained from the first label and the second label is distinguishable from a combined signal of said first and second labels.

10. A method for selecting an antigen, said method comprising:
providing a collection of antigens,
immunizing at least one animal with said collection of antigens, and
selecting an antigen for which clonal expansion of cells of a B-cell lineage-specific for a proteinaceous molecule has occurred.

11. The method according to claim 10, wherein said antigen comprises modified peptides derived from at least part of a proteinaceous molecule such that said modified peptides are immunologically different from any peptide of such proteinaceous molecule.

12. The method according to claim 10, further comprising selecting an antigen for which clonal expansion of antigen-specific immune cells is more extensive than the clonal expansion with another antigen of said collection.

13. The method according to claim 10, wherein clonal expansion of cells of the B-cell lineage is detected through a method comprising:
generating a synthetic peptide derived from at least part of said proteinaceous molecule,
immunizing an animal with said synthetic peptide, and
detecting in a sample obtained from the animal, a cell comprising a member of an affinity-binding pair that is specific for said proteinaceous molecule.

14. An isolated peptide, or pharmaceutically acceptable salt thereof, able to induce a cross-reactive immune response with a native protein, selected by the method according to claim 10.

15. The isolated peptide of claim 14, wherein the proteinaceous molecule is selected from the group consisting of GnRH, gastrin, hCG, and PTHrP derived from GnRH.

16. A composition comprising the isolated peptide or pharmaceutically acceptable salt thereof of claim 14 together with a pharmaceutically acceptable excipient.

17. A method of treating a subject believed to be suffering from a disease state, said disease state selected from the group consisting of prostate cancer, female GnRH-related cancer, gastrin-related colorectal cancer, gastric cancer, pancreatic cancer, hCG-related gynecological cancer, colorectal cancer, seminoma testicular cancer, bladder cancer, liver cancer, stomach cancer, pancreas cancer, lung cancer, brain cancer, kidney cancer, hCG-related ulcers, Duke's disease, cirrhosis, related cancer, PTHrP-related osteolytic bone disease, and PTHrP-related hypercalcaemia, said method comprising:
administering to the subject the composition of claim 16.

18. A method of blocking fertility in a subject, said method comprising:
administering the composition of claim 16 to the subject.

19. A process for identifying whether a collection of immune cells comprises a cell having at least two copies of a member of a specific binding pair, specific for an epitope in a proteinaceous molecule, said process comprising:
providing said collection with at least two molecules specific for said member of a specific binding pair, wherein a first of said molecules is associated with a first label and a second of said molecules is associated with a second label, and
determining whether said collection comprises cells stained by both labels, thus identifying a cell having at least two copies of the member of the specific binding pair, specific for the epitope in the proteinaceous molecule.

20. The process of claim 19, further comprising determining wherein the cell having at least two copies of a member of a specific binding pair, specific for an epitope in a proteinaceous molecule is a B-cell and isolating said B-cell.

21. A B-cell isolated by the process of claim 20.

22. A hybridoma cell line comprising the B cell of claim 21.

23. In a process for producing an antibody of the type using a B cell or a hybridoma, the improvement comprising:
using the hybridoma of claim 21 to produce the antibody.

24. An isolated antibody produced by the process of claim 23.

25. A composition comprising:
the isolated antibody of claim 24
together with a pharmaceutically acceptable excipient.

26. A kit of parts comprising:
at least a first molecule associated with a first label, and
at least a second molecule associated with a second label,
wherein said first and second molecules comprise at least two binding regions for a particular member of affinity-binding pair specific for an epitope in a proteinaceous molecule.

27. A method for selecting an antigen, said method comprising:
using the kit of parts according to claim 26 for selecting the antigen.

28. A method for detecting a cell in a collection of cells, said method comprising:
providing said collection with a first molecule and a second molecule able to specifically bind said cell, wherein said first molecule and said second molecule comprise different labels and further wherein said first and said second molecules comprise at least four binding sites for the cell, and
detecting a cell comprising both labels.

29. The method according to claim 28, wherein said cell comprises both labels at approximately equimolar amounts.

30. The method according to claim 28, wherein said cell is an immune cell.

31. The method according to claim 30, wherein said cell is obtained from an animal immunized with a molecule comprising said binding site or a functional derivative thereof.

32. The method according to claim 31, wherein the animal is immunized with a functional derivative that is similar, but not identical, to a part of a native protein or peptide, and wherein said binding is identical to said part of said native protein or peptide.

33. The method according to claim 32, wherein said functional derivative comprises an element selected from the group consisting of a dimer peptide, a tandem-dimer peptide, a mimotope of said antigen, and from 1 to 6 amino acids per 20 amino acids substituted by a D-amino acid and/or an L-alanine.
